(19) United States
(12) Patent Application Publication
(10) Pub. No.: US 2023/0366791 A1
(43) Pub. Date: Nov. 16, 2023

(54) ALL-OPTICAL VISUALIZATION OF SPECIFIC MOLECULES IN THE ULTRASTRUCTURAL CONTEXT OF BRAIN TISSUE

(71) Applicant: Yale University, New Haven, CT (US)

(72) Inventors: Ons M'Saad, New Haven, CT (US); Joerg Bewersdorf, New Haven, CT (US)

(21) Appl. No.: 18/295,446

(22) Filed: Apr. 4, 2023

Related U.S. Application Data
(60) Provisional application No. 63/362,426, filed on Apr. 4, 2022.

(51) Int. Cl. G01N 1/30; G01N 33/533

(52) U.S. Cl. CPC G01N 1/30; G01N 33/533; G01N 2001/305

(57) ABSTRACT

One aspect of the invention provides a method for preparing a biological sample. The method includes: exposing the biological sample to a photocrosslinkable substance adapted to bind to the biological sample, thereby forming a plurality of sample-photocrosslinkable-substance conjugates; embedding the sample in a hydrogel; and exposing the sample to light energy at a wavelength and intensity sufficient to cross-link the photocrosslinkable substance to the hydrogel.

### CROSS-REFERENCE TO RELATED APPLICATION(S)

This application claims the benefit of priority under 35 U.S.C. § 119(e) to U.S. Provisional Patent Application Ser. No. 63/362,426, filed Apr. 4, 2022. The entire content of this application is hereby incorporated by reference herein.

### BACKGROUND OF THE INVENTION

Understanding the molecular anatomy and neural connectivity of the brain requires imaging technologies that can map the 3D nanoscale distribution of specific proteins in the context of brain ultrastructure. Light and electron microscopy enable visualization of either specific labels or anatomical ultrastructure, but combining molecular specificity with anatomical context is challenging.

### SUMMARY OF THE INVENTION

One aspect of the invention provides a method for preparing a biological sample. The method includes: exposing the biological sample to a photocrosslinkable substance adapted to bind to the biological sample, thereby forming a plurality of sample-photocrosslinkable-substance conjugates; embedding the sample in a hydrogel; and exposing the sample to light energy at a wavelength and intensity sufficient to cross-link the photocrosslinkable substance to the hydrogel.

This aspect of the invention can have a variety of embodiments. The photocrosslinkable substance can be a sphingosine. The photocrosslinkable substance can be pacSph. The photocrosslinkable substance can include diazirine. The photocrosslinkable substance can include one or more selected from the group consisting of: a reporter group, a Clickable reporter group, and an alkyne reporter group.

The photocrosslinkable substance can be of X-Y-Z format wherein X can bind a biological substance, Y is a photocrosslinkable group, and Z is a reporter group.

The light energy can be UV light. The light energy can have a wavelength between 200 nm and 600 nm, (e.g., about 365 nm).

The hydrogel can be swellable. The hydrogel can be swellable and the hydrogel can be modified by inserting additional molecules into the hydrogel to increase its size. The sample can be photocrosslinked after embedding in a hydrogel. The sample is photocrosslinked before polymerization of the hydrogel. The sample can be photocrosslinked 5 while being embedded in a hydrogel. The sample can be photocrosslinked to one or more of a first and a second hydrogel network in a double-network hydrogel. The sample can be photocrosslinked to one or more of a first and/or nth hydrogel network in a multiple-network hydrogel, wherein n is a positive integer greater than 2.

The photocrosslinkable substance can bind to proteins. The photocrosslinkable substance can include an amine reactive group. The photocrosslinkable substance can include an NHS ester group.

The photocrosslinkable substance can bind to nucleic acids. The photocrosslinkable substance can bind to lipids.

The photocrosslinkable substance can include a neutral or positively charged lipid. The photocrosslinkable substance can include a ceramide.

The photocrosslinkable substance can bind to carbohydrates.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a pan-ExM-t workflow for mouse brain tissue sections. Panels (a)-(f) depict an experimental workflow. Panel (g) depicts a timeline summarizing the protocol. Abbreviations: FA: formaldehyde; AAm: acrylamide; NaOH: sodium hydroxide; DHEBA: N,N′-(1,2-dihydroxyethylene)bis-acrylamide; SDS: sodium dodecyl sulfate; PBS-T: 0.1% (v/v) TX-100 in PBS; ROI: region of interest.

FIG. 2 depicts synapse ultrastructure in dissociated neurons revealed by pan-ExM. Panel (a) depicts NHS ester pan-stained dendrite in a non-expanded sample showing dendritic spines. Panel (b) depicts axial view model of a synapse showing dense projections (DPs) and synaptic vesicles (SVs) in the presynaptic bouton, and the postsynaptic density (PSD), and the active zone (AZ) in the postsynaptic dendritic spine. Panel (c) depicts a top view of a synapse showing hexagonal dense projections (DPs) in the presynaptic bouton and synaptic vesicle (SV) attachment sites. Panels (d) and (f)-(k) depict pan-ExM-processed and NETS-ester-pan-stained spines including cupboard (h, j), stubby (f), and thin (k) shapes. Panel (e) depicts NHS-ester-pan-stained synapse showing hexagonally arranged DPs. Panel (1) is a transmission EM (TEM) image of a phosphotungstic acid (PTA) stained synapse showing prominent DPs (downward arrows) and a PSD (upward arrow). Panel (m) depicts pan-ExM processed and NHS ester pan-stained synapse for comparison, showing similar hallmark ultrastructural features. Panel (n) depicts DP-PSD distances (n=44 measurements from 4 independent experiments). Panel (o) depicts DP-DP distances (n=78 measurements from 6 independent experiments). Panel (p) depicts a comparison of Bassoon-PSD and DP-PSD distances (n=50 measurements from 3 independent samples). Panel (q) depicts a comparison of Homer1-PSD and DP-PSD distances (n=85 measurements from 4 independent samples). Panel (r) depicts a comparison of PSD95-DP and DP-PSD distances (n=25 measurements from 2 independent samples). *: p<0.05; **: p<0.01. Panel (s) depicts a relative spatial distributions of Bassoon, PSD-95, and Homer1 along the trans-synaptic axis. Panels (t), (w), and (z) depict axial (t, w) and top (z) views of synapses pan-stained with NHS ester. Panels (u), (x), and (aa) depict Bassoon immunolabeling of the same areas. Panels (v), (y), and (bb) depict respective overlays. Panels (cc)-(kk), (ll)-(qq), (rr)- (ww), and (xx)-(ccc) are the same as (t)-(bb) in samples labeled for Homer1, labeled for PSD-95, double-labeled for Homer1 and Bassoon, and labeled for Synaptophysin (SYN), respectively. The inset in (ccc) shows SYN puncta, representing synaptic vesicles, intercalated between neighboring DPs. Panel (ddd) depicts NHS ester image of a mitochondrion in a hippocampal rat neuron. Panel (eee) depicts SYTOX Green (SYX) staining of the same area. Panel (fff) depicts an overlay. Panel (ggg) depicts an NHS ester image of a nuclear pore complex (NPC). Panel (hhh) depicts an overlay of panel (ggg) with a SYTOX Green image of the same area. Panel (iii) depicts an NHS ester image of basal bodies in a mouse neuron. The inset shows the familiar centriolar cartwheel structure. Panel (jjj) depicts an NHS ester image of a cilium in a mouse neuron. Downward and upward arrows point to the basal body and ciliary tip, respectively. Gamma corrections: (d, e) γ=0.8; (h, j, k, m, cc, ll, oo, rr, uu, xx, aaa, iii, jjj) γ=0.7; (i) γ=0.6. jjj is a z-projection (intensity average) of 5 images. All scale bars are corrected for the expansion factor. Scale bars: (a) 800 nm, (d-m, t-ccc, jjj) 200 nm, (ddd-fff) 300 nm, (ggg, hhh) 50 nm, (iii) 100 nm.

FIG. 3 depicts how pan-ExM-t reveals mouse brain tissue ultrastructure. Panel (a) depicts NHS ester pan-stained tissue section of the mouse cortex. Panels (b) and (c) depict magnified areas in the black and dotted black boxes in a, respectively. Panels (d)-(h) depict magnified areas identified by the correspondingly colored boxes in (b) and (c) showing putative excitatory synapses. Panel (i) depicts linear expansion factor (n =254 measurements from 3 independent experiments). Panel (j) depicts ECS+lipid membrane (ECS+) fraction (n=14 measurements from 3 independent experiments). Panel (k) depicts image of neuropil in the hippocampus. Panel (l) depicts the same area as (k) where white pixels represent the ECS+. Panels (m)-(cc) depict putative excitatory synapses defined by a prominent PSD. Lime arrows in (n) and (p) point to mitochondria in the presynaptic bouton and in the postsynaptic compartment, respectively. The salmon arrow in (q) points to a spine neck. In panels (dd)-(ii), putative spine apparatuses (pink arrows) in the postsynaptic compartment defined by a characteristic lamellar arrangement. In panels (jj)-(ll), classification of synapses based on the patterns and intensity of the PSD; in panel (jj), class 1: the PSD is prominent and macular; in panel (kk), class 2: the PSD is prominent and perforated; in panel (ll), class 3: the PSD, unlike DPs, is barely visible. In panels (mm)-(vv), NHS ester pan-stained multisynaptic boutons and their postsynaptic partners. In panel (ww), lateral view of a centriole showing distal and proximal ends as well as distal appendages (turquoise arrows). In panel (xx) and (yy), top views of centrioles show the cartwheel structure (xx) and the 9-fold symmetry of microtubule triplets (yy). In panels (zz)-(ooo), mitochondria with vesicular cristae (zz-ccc, ggg, iii), lamellar cristae (ddd-fff), and teardrop-shaped with tubular extensions (yellow arrows) (mmm-ooo) are depicted. Salmon arrow in mmm points to putative myelinated sheaths. In panel (ppp), brain capillary showing endothelial cells (lavender arrow heads), putative tight junctions (TJs; teal arrows) that link neighboring endothelial cells, putative pericyte branch (salmon arrow), and the basement membrane (BM; yellow arrow) are visible. Gamma corrections: (a, ww-yy, ppp) γ=0.7; (m-vv, zz-ooo) γ=0.8. All scale bars are corrected for the expansion factor. Scale bars, (a) 5 μm, (b, c, ppp) 1 μm, (d-h, m-cc, jj-vv, xx, ww, zz-ccc, ggg, jjj, lll) 200 nm, (k, l) 3 μm, (dd-ii, xx, yy, ddd-fff, iii, nnn) 100 nm, (hhh, mmm, ooo) 400 nm.

FIG. 4 depicts how pan-ExM-t is compatible with antibody labeling of synaptic proteins in brain tissue. In panel (a), NHS ester (grayscale) pan-stained and Bassoon (red) immunolabeled brain tissue section. Panel (b) depicts a magnified area in the box in (a) showing a synapse. Panel (c) depicts Bassoon immunolabeling of the same area as in (b). Panel (d) depicts NHS ester pan-staining of the same area as in (b). Panel (e)-(p) depict additional examples similar to (b)-(d) showing lateral (e-m) and top (n-p) views of Bassoon-immunolabeled synapses. Panels (q)-(s) depict lateral views of a Class 3 synapse pan-stained with NHS ester and immunolabeled with Bassoon antibody indicating that Class 3 synapses are positive for Bassoon protein. Panel (t) depicts NHS ester (grayscale) pan-stained and Homer1 (magenta) immunolabeled brain tissue section. Panel (u) depicts magnified area in the magenta box in (t) showing a synapse. Panel (v) depicts Homer1 immunolabeling of the same area as in (u). Panel (w) depicts NHS ester pan-staining of the same area as in (u). Panels (x)-(ii) depict additional examples similar to (u)-(w) showing lateral (x-ff) and top (gg-ii) views of Homer1-immunolabeled synapses. Panels (jj)-(ll) depict lateral view of a Class 3 synapse pan-stained with NHS ester and immunolabeled with Homer1 antibody indicating that Class 3 synapses are negative for Homer1 protein. Panel (mm) depicts NHS ester (grayscale) pan-stained and PSD-95 (cyan) immunolabeled brain tissue section. Panel (oo) depicts magnified area in the cyan box in mm showing a synapse. Panel (pp) depicts PSD-95 immunolabeling of the same area as in (oo). Panel (qq) depicts NHS ester pan-staining of the same area as in (oo). Panels (rr)-(ccc) depict additional examples similar to (oo)-(qq) showing lateral (rr-zz) and top (aaa-ccc) views of PSD-95-immunolabeled synapses. Panels (ddd)-(fff) depict lateral views of a Class 3 synapse pan-stained with NHS ester and immunolabeled with PSD-95 antibody indicating that Class 3 synapses are negative for PSD-95 protein. Panel (ggg) depicts NHS ester (grayscale) pan-stained and Synaptophysin (SYN; yellow) immunolabeled brain tissue section. Panel (hhh) depicts a magnified area in the box in (ggg) showing a synapse. Panel (iii) depicts synaptophysin immunolabeling of the same area as in (hhh). Panel (jjj) depicts NHS ester pan-staining of the same area as in (hhh). Panels (lll)-(nnn) depicts an additional example similar to (hhh)-(jjj). Panel (ooo) depicts a comparison between Bassoon-PSD and DP-PSD distances (n=74 measurements from 3 fields of view (FOVs) in one independent experiment). Panel (ppp) depicts a comparison between Homer1-PSD and DP-PSD distances (n=120 measurements from 5 FOVs in 2 independent experiments). Panel (qqq) depicts a comparison between PSD95-DP and DP-PSD distances (n=113 measurements from 3 FOVs in one independent experiment). ****: p<0.0001. rrr, relative spatial distributions of Bassoon, PSD-95, and Homer1 along the trans-synaptic axis. All images (with the exception of ggg-nnn) are z-projections (intensity average) of 2 images. All NHS ester images were Gamma-corrected with y=0.7 with the exception of the Synaptophysin images (y=0.6). All scale bars are corrected for the expansion factor. Scale bars (a, t, mm) 2 μm, (b-m, q-s, u-ff, jj-ll, oo-zz, ddd-fff, hhh-nnn) 200 nm, (n-p, gg-ii, aaa-ccc) 100 nm, (ggg) 1 μm.

FIG. 5 depicts that pan-ExM-t is compatible with antibody labeling of brain tissue structural markers. Panel (a) depicts cortical neurons pan-stained with NHS ester (grayscale) and stained with SYTOX green (teal) showing a neuron nucleus. Panels (b)-(d) depict magnified view of the area in the green box in a and the SYTOX Green (c) and NHS ester (d) channels shown separately. Panel (e) depicts astrocyte pan-stained with NHS ester (grayscale) and immunolabeled with glial fibrillary acidic protein antibody (α-GFAP; magenta). Panel (f)-(h) depict a magnified view of the area in the magenta box in (e) showing GFAP filaments surrounding mitochondria, and the α-GFAP (g) and NHS ester (h) channels shown separately. Panel (i) is a 3D rendering of α-GFAP (magenta), SYTOX Green (green), and NHS ester (grayscale). Panels (j) and (k) are 3D renderings of α-GFAP (magenta) and blood vessels (BV) pan-stained with NHS ester (grayscale). Panel (l) depicts multiciliated ependymal epithelia pan-stained with NHS ester (grayscale). The inset shows 9-fold symmetry of cilia. Panels (m-r) depict lateral (m-o) and top (p-r) views of axons pan-stained with NHS ester (grayscale) and immunolabeled with myelin basic protein antibody (α-MBP; red). α-MBP (n, q) and NHS ester (o, r) channels of the areas shown in (m) and (p). Panel (s) is a 3D rendering of anti-MBP showing multiple axons. Panels (t) and (u) are neuropil pan-stained with NHS ester (grayscale) and anti-GFP (green) showing that only a sparse subset of cell bodies and neurites express GFP-Thy1. Panels (v-x) depicts the magnified area in the black box in (t) showing a neurite pan-stained with NHS ester (x) and immunolabeled with anti-GFP (w) where GFP-Thy1 is not expressed inside mitochondria. In (y-gg), a dendritic spine (y-aa) and axonal boutons (bb-gg) pan-stained with NHS ester and immunolabeled with anti-GFP. Panels (z), (cc), and (ff) depict anti-GFP channels of the areas shown in (y), (bb), and (ee), respectively. Panels (aa), (dd), and (gg) depict NHS ester channels of the areas shown in (y), (bb), and (ee), respectively. Panel (hh) depicts a 3D representation of a surface-rendered dendrite immunolabeled with anti-GFP (green) and NHS ester pan-staining (grayscale). Panel (ii) depicts 3D rendering of a dendrite immunolabeled with anti-GFP (green) and NHS ester pan-staining (grayscale). Gamma corrections: (a, e, m, p, t, u, x, aa, dd, gg) y=0.7. 3D renderings were processed with Imaris with gamma corrections applied. 3D image processing details for images i-l, s, hh, and ii are found in the Methods section. Scale bars in the 3D renderings i-l, s, hh, and ii are not corrected for the expansion factor. All other scale bars are corrected for the expansion factor. Scale bars (a) 5 μm, (b-d, e, m-o, u) 2 μm, (f-h, v-x) 500 nm, (p-r, t) 1 μm, (y-gg) 200 nm.

FIG. 6 depicts that pacSph enables lipid membrane pan-staining of brain tissue in pan-ExM-t. Panels (a-c) depict perikaryon pan-stained both with NHS ester (a) and pacSph (b) (overlay shown in c). Panels (d-l) depict magnified areas of the solid boxes in (a-c) showing the two sides of ER tubules (magenta arrow; d-f), the dashed boxes in (a-c) showing a mitochondrion (green arrow) in contact with the ER (magenta arrow; g-i), and the dotted boxes in a-c showing the nuclear envelope (j-l). Panel (m) depicts mitochondria signal levels in tissue pan-stained with pacSph and not irradiated with UV (white; n=35 measurements from 5 FOVs in one independent experiment); pan-stained with pacSph and irradiated with UV before hydrogel embedding (green; n=37 measurements from 5 FOVs in one independent experiment); and pan-stained with pacSph and irradiated with UV after hydrogel embedding (pink; n=39 measurements from 5 FOVs in one independent experiment). ****: p<0.0001. Panel (n) depicts chemical structure of pacSph. The probe is photocrosslinked via its diazirine group (magenta) and labeled via its alkyne group (green). Panels (o-t) depict synapses pan-stained both with NHS ester (o, r) and pacSph (p, s) (overlays shown in q, t). Panels (u-w) depict neuropil pan-stained both with NHS ester (u) and pacSph (v) (overlay shown in w). Panels (x-cc) depict magnified areas of the solid (x, z, bb) and dashed (y, aa, cc) boxes in u-w showing synapses. Panels (dd-oo) depict neurites and synapses pan-stained both with NHS ester (dd, gg, jj, mm) and pacSph (ee, hh, kk, nn) (overlays shown in ff, ii, ll, oo). Panels (pp-rr) depict brain capillary pan-stained both with NHS ester (pp) and pacSph (qq) (overlay shown in rr). Arrows point to mitochondria. Gamma corrections: (a) y=0.7; (p, r, u, dd, gg, jj, mm, pp) γ=0.5. All scale bars are corrected for the expansion factor. Scale bars (a-c) 3 μm, (d-i, dd-ii) 500 nm, (u-w) 2 μm, (j-l, o-t, x-cc, jj-oo) 250 nm, (pp-rr) 1 μm.

FIG. 7 depicts a method of preparing a biological sample according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

The terms “proximal” and “distal” can refer to the position of a portion of a device relative to the remainder of the device or the opposing end as it appears in the drawing. The proximal end can be used to refer to the end manipulated by the user. The distal end can be used to refer to the end of the device that is inserted and advanced and is furthest away from the user. As will be appreciated by those skilled in the art, the use of proximal and distal could change in another context, e.g., the anatomical context in which proximal and distal use the patient as reference, or where the entry point is distal from the user.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Embodiments of the invention present pan-Expansion Microscopy of tissue (pan-ExM-t), an all-optical mouse brain imaging method that combines ˜24-fold linear expansion of biological samples with fluorescent pan-staining of protein densities (providing ultrastructural context), and immunolabeling of protein targets (for molecular imaging). Applicant demonstrates the versatility of this approach by imaging established markers Homer1, Bassoon, PSD-95, Synaptophysin, GFAP, MBP and anti-GFP antibodies in dissociated neuron cultures and mouse brain tissue sections. pan-ExM-t reveals these markers in the context of ultrastructural features such as pre and postsynaptic densities, 3D nanoarchitecture of neuropil, and the fine structures of cellular organelles. pan-ExM-t is adoptable in any neurobiological laboratory with access to a confocal microscope and has therefore broad applicability in the research community.

### Introduction

Three-dimensional microscopy techniques are instrumental to understanding of brain organization with its complex morphology, spanning from the sub-synapse scale to neural circuitry maps. Despite significant advances in imaging, no microscopy method can provide a detailed molecular topography of the synapse. While electron microscopy (EM) is the gold standard for ultrastructural analysis, localizing specific proteins still relies on immunogold labeling or electron-dense peroxidase substrates, neither of which are reliable nor quantitative. Fluorescence microscopy, on the other hand, enables highly specific, multicolor labeling of proteins of interest. However, it fails to reveal the underlying ultrastructural context. Even with the advent of super-resolution microscopy, where single molecules can be imaged in 3D with spatial resolutions down to ˜10 nm, delineating context is unattainable: fluorescent dyes are comparatively bulky (˜1 nm) and susceptible to quenching when densely packed, hindering imaging of crowded biomolecules.

Expansion Microscopy (ExM), which physically expands tissue samples ˜4 to 20-fold by embedding them in swellable hydrogels, has resolved subcellular protein distributions of synaptic targets in mouse brain tissue, as well as map RNA transcripts within axons and dendrites in the brain. However, while the expansion-related decrowding avoids fluorescence quenching, ExM has traditionally capitalized almost exclusively on antibody labelings, and no ExM method has demonstrated correlative molecular and contextual imaging with synaptic resolution so far. Correlative light and electron microscopy (CLEM) has been the only demonstrated option aligning specific molecular markers with sample ultrastructure. However, because of its operational complexity and challenge to label endogenous proteins specifically, especially in 3D imaging, its application has been very limited.

Applicant recently discovered a new principle for an optical contrast equivalent to EM heavy-metal stains, which allows for ultrastructural analysis using conventional light microscopy. By physically expanding a biological sample ˜20-fold in every dimension without losing the protein content, bulk staining of proteins can now reveal the totality of subcellular compartments down to size scales of ˜15 nm. Applicant calls this approach pan-ExM, referencing the philosophy of labeling the whole (Greek: pan) and the original concept of sample expansion in ExM. The 20³=8,000-fold volume expansion decrowds the cellular environment and thereby overcomes the permeability, quenching, and sampling limitations associated with bulk staining of conventional samples. This approach allows for conventional light microscopes to acquire EM-like images of adherent monolayer cells, revealing subcellular features such as mitochondria cristae and Golgi cisternae by their anatomical characteristics.

The expansion mechanism in pan-ExM differs from other ExM approaches in that the sample expands ˜20-fold while proteins are retained in the hydrogel for subsequent staining. To achieve this, pan-ExM avoids protease digestion and instead uses polymer entanglement to expand sample components twice without losing them: by embedding an already expanded sample prepared with a cleavable crosslinker in a second dense superabsorbent hydrogel, entanglements between polymer chains of the first and final hydrogels can physically interlock protein-polymer hybrids in this latter polymer network, thereby preserving the proteome while iteratively expanding it. The conceptual advance in pan-staining of highly expanded samples takes light microscopy to the realm of ultrastructural context imaging and generates EM heavy-metal stain-like images. In combination with well-established labeling methods in fluorescence microscopy, pan-staining provides nanoscale context to proteins of interest, analog to CLEM.

Here, Applicant introduces pan-ExM-t, a pan-ExM protocol that enables contextual imaging of thick mouse brain tissue sections. Analogous to EM, Applicant discovered that hallmark ultrastructural features such as pre- and postsynaptic densities can be identified by their morphological characteristics, allowing, for the first-time, ultrastructural imaging of putative synapses by light microscopy without specific labels. The developments presented herein give every neurobiologist the power to perform routine 3D pan-ExM-t imaging of brain tissue sections using their standard confocal microscope.

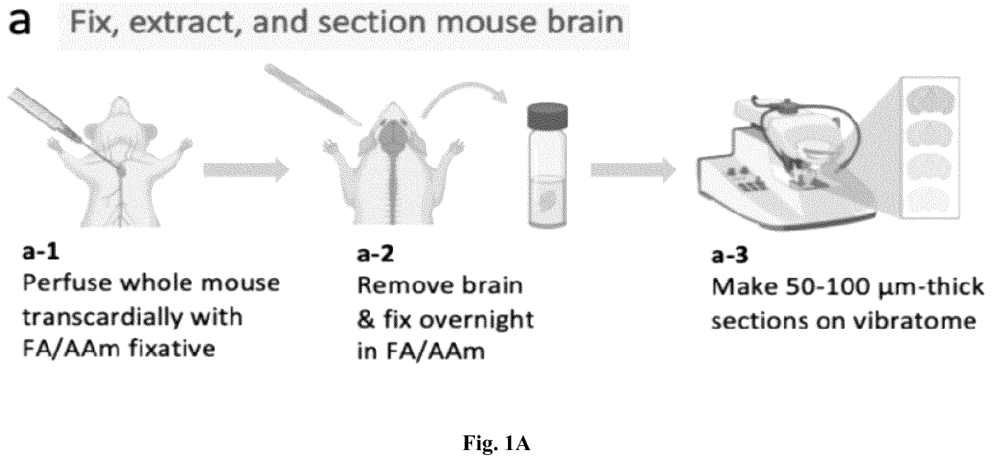

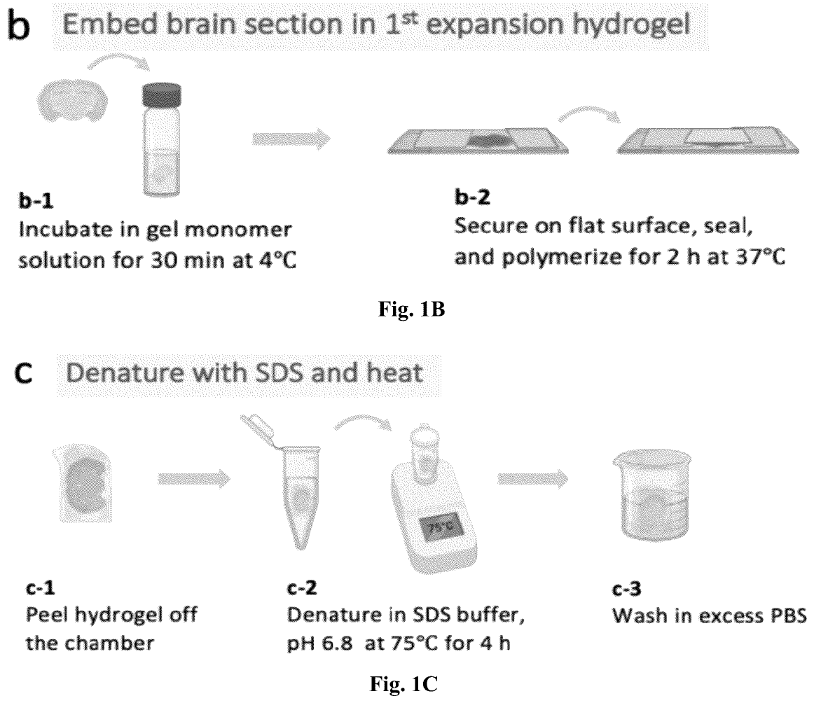

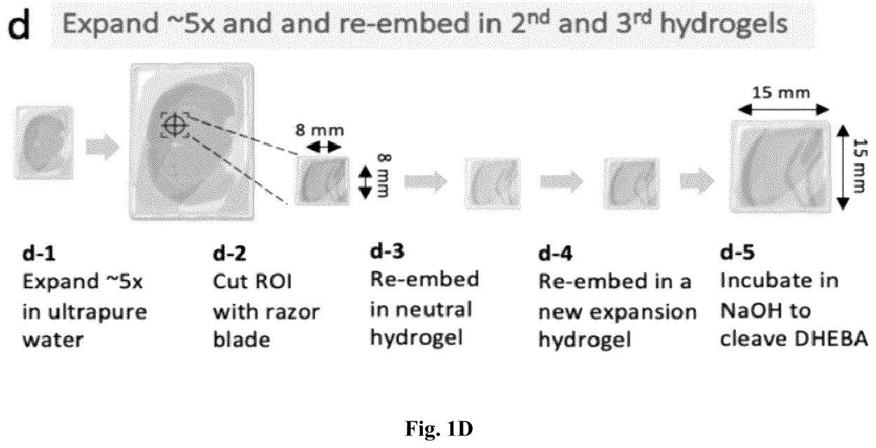

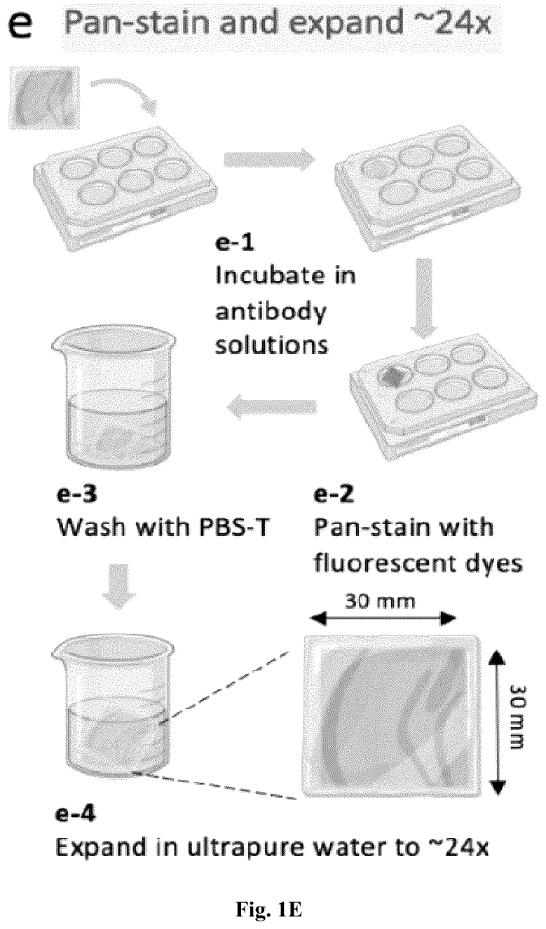

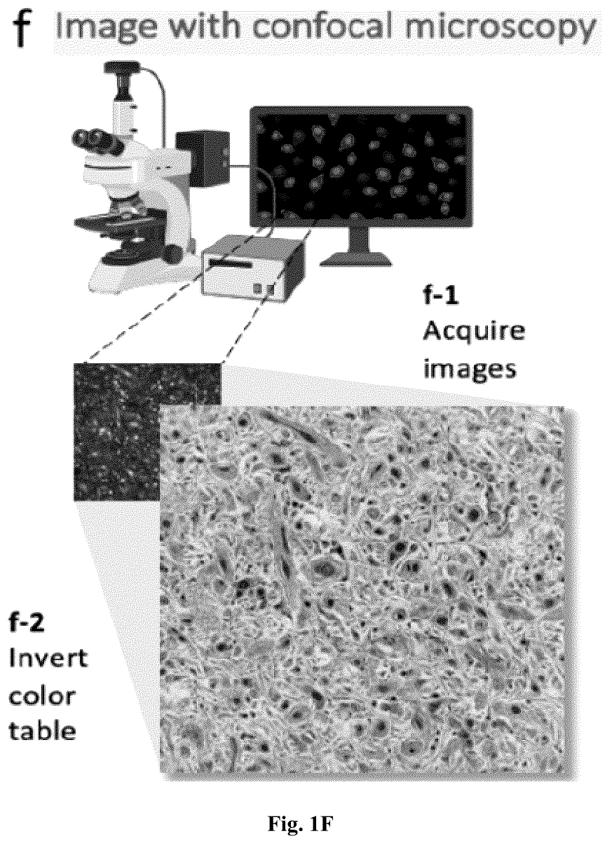

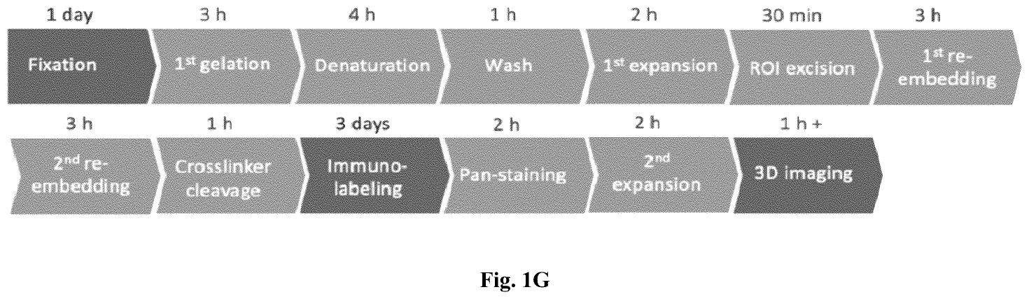

FIG. 1 shows an overview of the novel brain-tissue pan-ExM-t protocol. In brief, mice are transcardially perfused with fixative containing both formaldehyde (FA) and acrylamide (AAm) and their brains are extracted surgically and incubated in the same fixative overnight at 4° C. The brains are then sectioned at 50-100 μm thickness using a vibratome and stored in PBS until future use (FIG. 1, Panel a). Each tissue section to be expanded is embedded in a dense poly(acrylamide/sodium acrylate) co-polymer that is cross-linked with N,N′-(1, 2-dihydroxyethylene)bisacrylamide (DHEBA), an acrylamide crosslinker with a cleavable amidomethylol bond (FIG. 1, Panel b). After polymerization, the now tissue-hydrogel hybrid is denatured with sodium dodecyl sulfate (SDS) in heated buffer (pH 6.8) for 4 hours (FIG. 1, Panel c) and expanded ˜5-fold in deionized water (FIG. 1, Panel d, blue). Next, a specific region of interest (ROI, ˜8×8 mm2) is cut and re-embedded first in a neutral polyacrylamide hydrogel cross-linked with DHEBA (FIG. 1, panel d, green) and then in a poly(acrylamide/sodium acrylate) co-polymer cross-linked with N,N′-methylenebis(acrylamide) (BIS), a non-hydrolysable acrylamide crosslinker (FIG. 1, panel d, orange). No secondary fixation of proteins before the re-embedding is required. The sample is then incubated in 200 mM sodium hydroxide to cleave DHEBA so that the crosslinked first and second hydrogel polymer networks are disentangled. After neutralization with multiple PBS washing steps, the sample is labeled with antibodies, pan-stained with fluorescent dyes, washed with detergents, and expanded to its final size of ˜24-fold in ultrapure water (FIG. 1, panel e). The expanded (and as a side-effect: optically cleared) sample is finally imaged on a standard confocal microscope (FIG. 1, panel f) and can be stored at 4° C. for months.

### EXPERIMENTAL EXAMPLES

### Methods

Experimental model and subject details

Neuron culture

Rat neuron cultures used to generate NHS ester, anti-Homer1, anti-Bassoon, and anti-PSD-95 images were cultured as described previously.

Rat neuron cultures used to generate NHS ester and anti-Synaptophysin images were cultured as follows: hippocampal CA3-CA1 regions were isolated and dissected from E18 Sprague Dawley Rats (Charles River Laboratories) in the presence of chilled hibernate E media (BrainBits). Hippocampal neurons were then incubated at 37° C. for 25 minutes in 0.25% trypsin (Corning; catalog no. 25-053 CI) and immediately washed with DMEM (Thermofisher; catalog no. 11965-118) containing 10% fetal bovine serum (Thermofisher; catalog no. 16000044). Following trypsinization, hippocampi were triturated and immediately plated in DMEM containing 10% fetal bovine serum on poly-D-lysine (Thermofisher; catalog no. ICN10269491) coated 18 mm glass coverslips (Warner instruments; catalog no. 64-0734 [CS-18R17]). After 1 hour of incubation at 37° C., DMEM was removed and replaced with Neurobasal-A (Thermofisher; catalog no. 10888-022) medium containing 2% B-27 (Thermofisher; catalog no. 17504001) and 2 mM Glutamax (Gibco; catalog no. 35050061). Experiments were performed 15-17 days after plating.

Mouse hippocampal cultures used to generate 5-fold expansion images were performed on BalbC PO pups, in accordance with IACUC protocol number 2019-07912. Hypothermia was induced in pups using ice, followed by decapitation and hippocampal dissection. Hippocampi were dissociated using 200 units Papain (Worthington LS003124, 200 units) for 40 minutes at 37° C. Neurons were triturated and plated onto 12 mm Poly-D-lysine coated coverslips in a 24 well dish at a density of 150,000 cells per well in Neurobasal/B27 Plus media (Thermo-Fisher; catalog no. A3653401) supplemented with 10% FBS and 1% Penicillin/Streptomycin (Thermo-Fisher; catalog no. 15140163). Following 3 hours of incubation at 37° C. and 5% CO2, the media was replaced with Neurobasal/B27 Plus media (Thermo-Fisher; catalog no. A3653401) supplemented with 1% Penicillin/Streptomycin (Thermo-Fisher; catalog no. 15140163)). Fixation was performed at DIV 14-15.

Experimental models

Brain tissue experiments were conducted in 4 wild-type (C57BL/6) adult male mice and 1 Thy1-EGFP mouse (4-8 weeks), obtained from the Jackson Laboratory.

Method details

Neuron fixation

Neurons immunolabeled with Homer1, Bassoon, or PDS-95 were fixed with 4% formaldehyde in 1×PBS (Thermofisher; catalog no. 10010023) for 15 min at RT. Neurons immunolabeled with Synaptophysin were fixed with 3% FA and 0.1% glutaraldehyde in 1×PBS for 15 min at RT. Images showing nucleoids in mitochondria were from samples fixed with 3% FA+0.1% GA in lx PBS for 15 min at RT. Images showing centrioles and NPCs were from samples fixed with 4% FA in 1×PBS for 15 min at RT. After fixation, all samples were rinsed three times with lx PBS and processed according to the pan-ExM protocol immediately after.

Formaldehyde (FA; catalog no. 15710) and glutaraldehyde (GA; catalog no. 16019) were purchased from Electron Microscopy Sciences.

Mice perfusion and brain fixation

All experiments were carried out in accordance with National Institutes of Health (NIH) guidelines and approved by the Yale IACUC.

All wild-type (C57BL/6) adult male mice were between P30 and P35 and maintained in the vivarium with a 12-h light˜dark cycle, stable temperature at 22±1° C. and humidity between 20 and 50%. Mice were transcardially perfused first with ice-cold 1×PBS and then with either 4% FA, 4% FA+0.1% FA, or 4% FA+20% acrylamide (AAm; Sigma; catalog no. A9099) (all in 1×PBS, pH 7.4). Brains were isolated and post-fixed overnight in the same perfusion solution at 4° C. Brains were subsequently washed and stored in PBS at 4° C. Brains were mounted in ice-cold PBS and coronally sectioned at 70 μm using a vibrating microtome (Vibratome 1500, Harvard Apparatus). Medial hippocampal slices were selected and washed four times for 15 min in PBS at room temperature. Sections were stored in PBS at 4° C. for up 20 to 3 months.

Mouse was anesthetized with ketamine (100 mg/kg, Butler Schein: 9952949) and xylazine (10 mg/kg, Akorn: NDC 59399-110-20) in saline (Hudson RCI: 200-59). The mouse was transcardially perfused first with ice cold 1×PBS (Fisher; BP2944-100) and then with 4% FA (J. T. Baker; S898-07)+20% AAm in 1×PBS, pH 7.4. Brain was isolated and postfixed overnight in 4% FA+20% AAm in 1×PBS at 4° C. Brain was then washed in PBS and coronally sectioned at 70 μm using vibrating microtome (Leica VT1000, Leica Biosystems, Nussloch, Germany). Sections were stored in 1×PBS with 0.01% sodium azide (Sigma-Aldrich, St. Louis, MO) at 4° C. for up to 3 months.

Pan-ExM-t reagents

Acrylamide (AAm; catalog no. A9099), N,N′-methylenebis(acrylamide) (BIS; catalog no. 146072) were purchased from Sigma-Aldrich. N,N′-(1,2-Dihydroxyethylene)bisacrylamide (DHEBA) was purchased from Sigma-Aldrich (catalog no. 294381) and Santa Cruz Biotechnologies (catalog no. sc-215503), with the latter being of better purity. Sodium acrylate (SA) was purchased from both Sigma Aldrich (catalog no. 408220) and Santa Cruz Biotechnologies (catalog no. sc-236893C). To verify that SA was of acceptable purity, 38% (w/v) solutions were made in water and checked for quality as previously reported. Only solutions that were light yellow were used. Solutions that were yellow and/or had a significant precipitate were discarded. Solutions with a minimum precipitate were centrifuged at 4000 rpm for 5 min and the supernatant was transferred to a new bottle and stored at 4° C. until use. Ammonium persulfate (APS) was purchased from both American Bio (catalog no. AB00112) and Sigma-Aldrich (catalog no. A3678). N,N,N′,N′-tetramethylethylenediamine (TEMED; catalog no. AB02020) was purchased from American Bio. 10X phosphate buffered saline (10X PBS; catalog no. 70011044) was purchased from Thermofisher. Sodium Dodecyl Sulfate (SDS; catalog no. AB01922) was purchased from American Bio and Guanidine hydrochloride (G-HCl; catalog no. G3272) was purchased from Sigma-Aldrich

Pan-ExM gelation chamber for dissociated neurons

The gelation chamber was constructed using a glass microscope slide (Sigma-Aldrich, catalog no. 58400) and two spacers, each consisting of a stack of two no. 1.5 22×22 mm coverslips (Fisher Scientific, catalog no. 12-541B). The spacers were superglued to the microscope slide on both sides of the neuron-adhered coverslip, with this latter coverslip glued in between. A no. 1.5 22×22 mm coverslip was used as a lid after addition of the gel solution. This geometry yielded an initial gel thickness size of ˜170 μm.

Pan-ExM-t gelation chamber for brain tissue sections

The gelation chamber was constructed using a glass microscope slide (Sigma-Aldrich, catalog no. 58400) and two spacers, each consisting of one no. 1.5 22×22 mm coverslip (Fisher Scientific, catalog no. 12-541B). The spacers were superglued to the microscope slide spaced ˜1 cm from one another. After incubation in activated first expansion gel solution (as described below in First round of expansion for brain tissue sections), the 70 μm-thick brain tissue section was placed and flattened on an additional no. 1.5 22×22 mm coverslip which was used as a chamber lid after addition of more activated first expansion gel solution on the microscope slide in-between the spacers. This geometry yielded an initial gel thickness size of ˜170 μm.

First round of expansion for dissociated neurons

Neurons, previously fixed as described in the Neuron fixation section, were incubated in post-fixation solution (0.7% FA+1% AAm (w/v) in 1×PBS) for 6-7 h at 37° C. Next, the neurons were washed twice with 1×PBS for 10 min each on a rocking platform and embedded in the first expansion gel solution (18.5% (w/v) SA+10% AAm (w/v)+0.1% (w/v) DHEBA+0.25% (v/v) TEMED+0.25% (w/v) APS in 1×PBS). Gelation proceeded for 10-15 min at room temperature (RT) and then for 1.5 h at 37° C. in a humidified chamber. Coverslips with hydrogels were then incubated in ˜4 mL denaturation buffer (200 mM SDS+200 mM NaCl +50 mM Tris in MilliQ water, pH 6.8) in 6-well plates for 45 min at 37° C. Gels were then transferred to denaturation buffer-filled 1.5 mL Eppendorf tubes and incubated at 73° C. for 1 h.

Next, the gels were washed twice with PBS for 20 min each and stored in PBS overnight at 4° C. Gels were then cut and placed in 6-well plates filled with MilliQ water for the first expansion. Water was exchanged twice every 30 min and once for 1 h. Gels expanded between 4.0× and 4.5× according to SA purity (see pan-ExM-t reagents).

First round of expansion for brain tissue sections

Brain tissue, previously fixed and sectioned to 70 μm as described in the Brain perfusion section, were first incubated in inactivated first expansion gel solution (19% (w/v) SA+10% AAm (w/v)+0.1% (w/v) DHEBA in lx PBS) for 30-45 min on ice and then in activated first expansion gel solution (19% (w/v) SA+10% AAm (w/v) +0.1% (w/v) DHEBA +0.075% (v/v) TEMED+0.075% (w/v) APS in 1×PBS) for 15-20 min on ice before placing in gelation chamber. The tissue sections were gelled for 15 min at RT and 2 h at 37° C. in a humidified chamber. Next, the tissue-gel hybrids were peeled off of the gelation chamber and incubated in ˜4 mL denaturation buffer (200 mM SDS+200 mM NaCl+50 mM Tris in MilliQ water, pH 6.8) in 6-well plates for 15 min at 37° C. Gels were then transferred to denaturation buffer-filled 1.5 mL Eppendorf tubes and incubated at 75° C. for 4 h. The gels were washed twice with 1×PBS for 20 min each and once overnight at RT. Gels were optionally stored in 1×PBS at 4° C. The samples were then placed in 6-well plates filled with MilliQ water for the first expansion. Water was exchanged three times every 1 h. Gels expanded between ˜5× according to SA purity (see pan-ExM-t reagents).

Re-embedding in neutral hydrogel

Expanded hydrogels (of both dissociated neuron and tissue samples) were incubated in fresh re-embedding neutral gel solution (10% (w/v) AAm+0.05% (w/v) DHEBA+0.05% (v/v) TEMED+0.05% (w/v) APS) two times for 20 min each on a rocking platform at RT. Immediately after, the residual gel solution was removed by gentle pressing with Kimwipes.

Each gel was then sandwiched between one no. 1.5 coverslip and one glass microscope slide. Gels were incubated for 1.5 h at 37° C. in a nitrogen-filled and humidified chamber. Next, the gels were detached from the coverslips and washed two times with 1×PBS for 20 min each on a rocking platform at RT.

The samples were stored in 1×PBS at 4° C. No additional post-fixation of samples after the re-embedding step was performed.

Second round of expansion

Re-embedded hydrogels (of both dissociated neuron and tissue samples) were incubated in fresh second hydrogel gel solution (19% (w/v) SA+10% AAm (w/v)+0.1% (w/v) BIS +0.05% (v/v) TEMED+0.05% (w/v) APS in 1×PBS) two times for 15 min each on a rocking platform in 4° C. Each gel was then sandwiched between one no. 1.5 coverslip and one glass microscope slide. Gels were incubated for 1.5 h at 37° C. in a nitrogen-filled and humidified chamber. Next, to dissolve DHEBA, gels were detached from the coverslips and incubated in 200 mM NaOH for 1 h on a rocking platform at RT. Gels were afterwards washed three to four times with 1×PBS for 30 min each on a rocking platform at RT or until the solution pH reached 7.4. The gels were optionally stored in lx PBS at 4° C. Subsequently, the gels were labeled with antibodies and pan-stained with NHS ester dyes. Finally, the gels were placed in 6-well plates filled with MilliQ water for the second expansion. Water was exchanged at least three times every 1 h at RT. Gels expanded ˜4.5× according to SA purity (see pan-ExM-t Reagents) for a final expansion factor of ˜16× (dissociated neurons) and ˜24× (brain tissue).

Antibody labeling of neurons post-expansion

Samples immunolabeled with Homer1, Bassoon, and PSD-95 were fixed with 4% FA in 1×PBS for 15 min as described in the Neuron fixation section, processed with pan-ExM, and incubated overnight (˜15 h) with monoclonal anti-Homer1 antibody (abcam, catalog no. ab184955), monoclonal anti-Bassoon antibody (abcam, catalog no. ab82958), or monoclonal anti-PSD-95 antibody (antibodiescinc, catalog no. 75-028) diluted 1:500 in antibody dilution buffer (0.05% TX-100 30 0.05% NP-40+0.2% BSA in 1×PBS). All primary antibody incubations were performed on a rocking platform at 4° C. Gels were then washed with 0.1% (v/v) TX-100 in 1×PBS twice for 20 min each on a rocking platform at RT and once overnight (˜15 h) at 4° C. Next, samples were incubated overnight with ATT0594-conjugated anti-mouse antibodies (Sigma-Aldrich, catalog no. 76085) or ATT0594-conjugated anti-rabbit antibodies (Sigma-Aldrich, catalog no. 77671) diluted 1:500 in antibody dilution buffer. All secondary antibody incubations were performed on a rocking platform at 4° C. The gels were subsequently washed in 0.1% (v/v) TX-100 in 1×PBS twice for 20 min each at RT and once overnight at 4° C. Gels were stored in PBS at 4° C. until subsequent treatments. Bovine serum albumin (BSA; catalog no. 001-000-162) was purchased from Jackson ImmunoResearch and NP-40 (catalog no. T8786) and Triton X-100 (TX-100; catalog no. T8787) was purchased from Sigma-Aldrich.

Samples immunolabeled with both Homer1 and Bassoon simultaneously were fixed with 4% FA in 1×PBS for 15 min as described in the Neuron fixation section, processed with pan-ExM, and incubated overnight (˜15 h) with both monoclonal anti-Homer1 antibody (abcam, catalog no. ab184955) and monoclonal anti-Bassoon antibody (abcam, catalog no. ab82958) diluted 1:500 in antibody dilution buffer (0.05% TX-100+0.05% NP-40+0.2% BSA in 1×PBS). Primary antibody incubations were performed on a rocking platform at 4° C. Gels were then washed with 0.1% (v/v) TX-100 in 1×PBS twice for 20 min each on a rocking platform at RT and once overnight (˜15 h) at 4° C. Next, samples were incubated overnight (˜15 h) with both ATTO594-conjugated anti-rabbit antibodies (Sigma-Aldrich, catalog no. 77671) and ATTO647N-conjugated anti-mouse antibodies (Sigma-Aldrich, catalog no. 50185) diluted 1:500 in antibody dilution buffer. Secondary antibody incubations were performed on a rocking platform at 4° C. The gels were subsequently washed in 0.1% (v/v) TX-100 in 1×PBS twice for 20 min each at RT and once overnight at 4° C. Gels were stored in PBS at 4° C. until subsequent treatments.

Samples immunolabeled with Synaptophysin were fixed with 3% FA+0.1% GA 1×PBS as described in the Neuron fixation section, processed with pan-ExM, and incubated for ˜36-40 h at 37° C. with rabbit anti-synaptophysin (Cell signaling technology, catalog no. 36406) diluted 1:250 in antibody dilution buffer (2% (w/v) BSA in 1×PBS). Gels were then washed in PBS-T (0.1% (v/v) Tween 20 in 1×PBS) three times for 20 min each on a rocking platform at RT. Next, samples were incubated for ˜16-20 h at 37° C. with donkey anti-rabbit CF568 (Biotium, catalog no. 20098) diluted 1:250 in antibody dilution buffer. The gels were washed in PBS-T three times for 20 min each at RT each before subsequent pan-staining.

Antibody labeling of brain tissue samples post-expansion

Brain tissue samples immunolabeled with Homer1, Bassoon, PSD-95, and Synaptophysin were previously fixed with 4% FA+20% AAm in 1×PBS overnight at 4° C. and sectioned to 70 μm as described in the Brain perfusion section. Next, they were washed three times with 1×PBS for 30 min each on a rocking platform at RT and processed with the pan-ExM-t protocol with this modification: the monomer composition of the second expansion hydrogel was 10% (w/v) AAm+9% SA (w/v). Samples were subsequently incubated for ˜30 h with monoclonal anti-Homer1 antibody (abcam, catalog no. ab184955), monoclonal anti-Bassoon antibody (abcam, catalog no. ab82958), monoclonal anti-PSD-95 antibody (antibodiescinc, catalog no. 75-028), or anti-Synaptophysin antibody (SYSY, catalog no. 101 011) diluted 1:250 in antibody dilution buffer (0.05% TX-100+0.05% NP-40+0.2% BSA in 1×PBS). All primary antibody incubations were performed on a rocking platform at 4° C. Next, samples were incubated for ˜30 h with ATTO594-conjugated anti-mouse antibodies (Sigma-Aldrich, catalog no. 76085) or ATTO594-conjugated anti-rabbit antibodies (Sigma-Aldrich, catalog no. 77671) diluted 1:250 in antibody dilution buffer. Gels were then washed in 0.1% (v/v) TX-100 in 1×PBS four times for 30 min to 1 h each on a rocking platform at RT and once overnight at 4° C. All secondary antibody incubations were performed on a rocking platform at 4° C. The gels were subsequently washed in 0.1% (v/v) TX-100 in 1×PBS four times for 30 min to 1 h each on a rocking platform at RT and once overnight at 4° C. Gels were stored in PBS at 4° C. until subsequent treatments.

Brain tissue samples immunolabeled with GFAP, MBP, and GFP were previously fixed with 4% FA+20% AAm in lx PBS overnight at 4° C. and sectioned to 70 μm as described in the Brain perfusion section. Next, they were washed three times with 1×PBS for 30 min each on a rocking platform at RT and processed with the pan-ExM-t protocol. Samples were subsequently incubated for ˜30 h with polyclonal anti-GFAP antibody (Thermofisher, catalog no. PA1-10019), monoclonal anti-MBP (BioLegend, catalog no. 808401), or polyclonal anti-GFP (Thermofisher, catalog no. A-1122) diluted 1:500 in antibody dilution buffer (0.05% TX-100 +0.05% NP-40+0.2% BSA in 1>PBS). All primary antibody incubations were performed on a rocking platform at 4° C. Gels were then washed in 0.1% (v/v) TX-100 in 1×PBS four times for 30 min to 1 h each on a rocking platform at RT and once overnight at 4° C. Next, samples were incubated for ˜30 h with ATTO594-conjugated anti-mouse antibodies (Sigma-Aldrich, catalog no. 76085) or ATTO594-conjugated anti-rabbit antibodies (Sigma-Aldrich, catalog no. 77671) diluted 1:500 in antibody dilution buffer. Gels were then washed in 0.1% (v/v) TX-100 in 1×PBS four times for 30 min to 1 h each on a rocking platform at RT and once overnight at 4° C. All secondary antibody incubations were performed on a rocking platform at 4° C. The gels were subsequently washed in 0.1% (v/v) TX-100 in 1×PBS four times for 30 min to 1 h each on a rocking platform at RT and once overnight at 4° C. Gels were stored in PBS at 4° C. until subsequent treatments.

NHS ester pan-staining of neurons

After antibody labeling, gels were incubated for 1.5 h with 20 μg/mL NHS ester-ATTO532 (Sigma-Aldrich, catalog no. 88793) (Homer1, Bassoon, PSD-95, centriole, and NPC samples), or 20 μg/mL NHS ester-ATTO488 (Sigma-Aldrich, catalog no. 41698) (Synaptophysin samples), or 20 μg/mL NHS ester-ATTO594 (Sigma-Aldrich, catalog no. 08741) (mitochondria samples), dissolved in 100 mM sodium bicarbonate solution (Sigma-Aldrich, catalog no. SLBX3650) on a rocking platform at RT. The gels were subsequently washed four to six times in either lx PBS or PBS-T for 20 min each on a rocking platform at RT.

NHS ester pan-staining of brain tissue sections

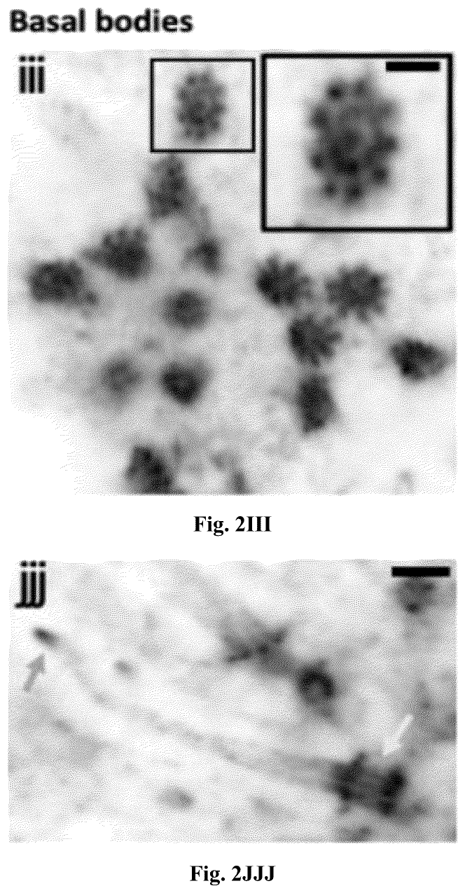

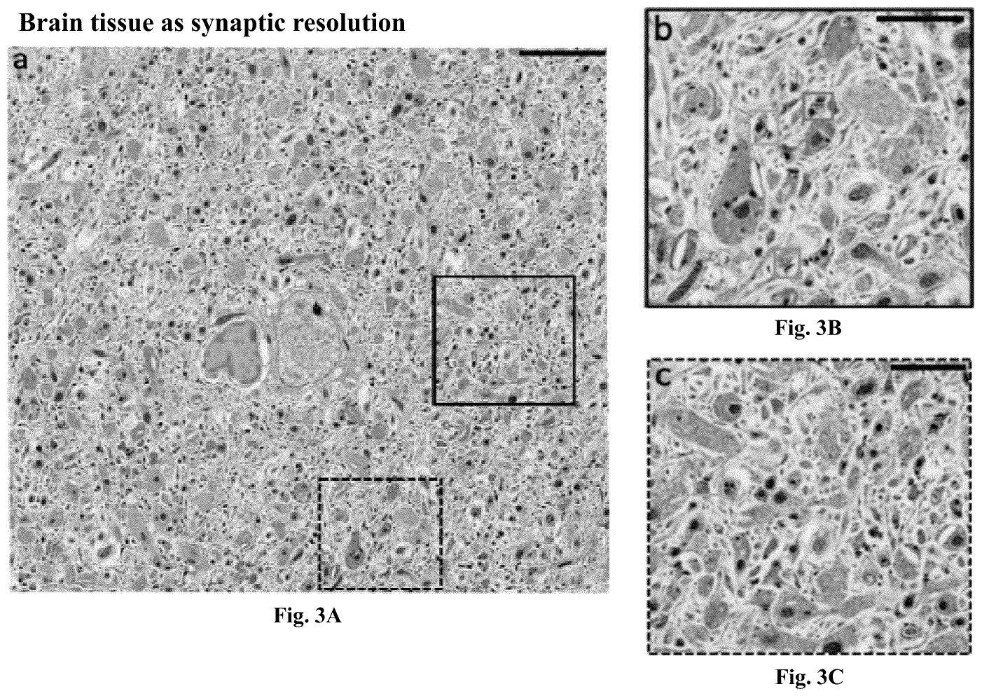

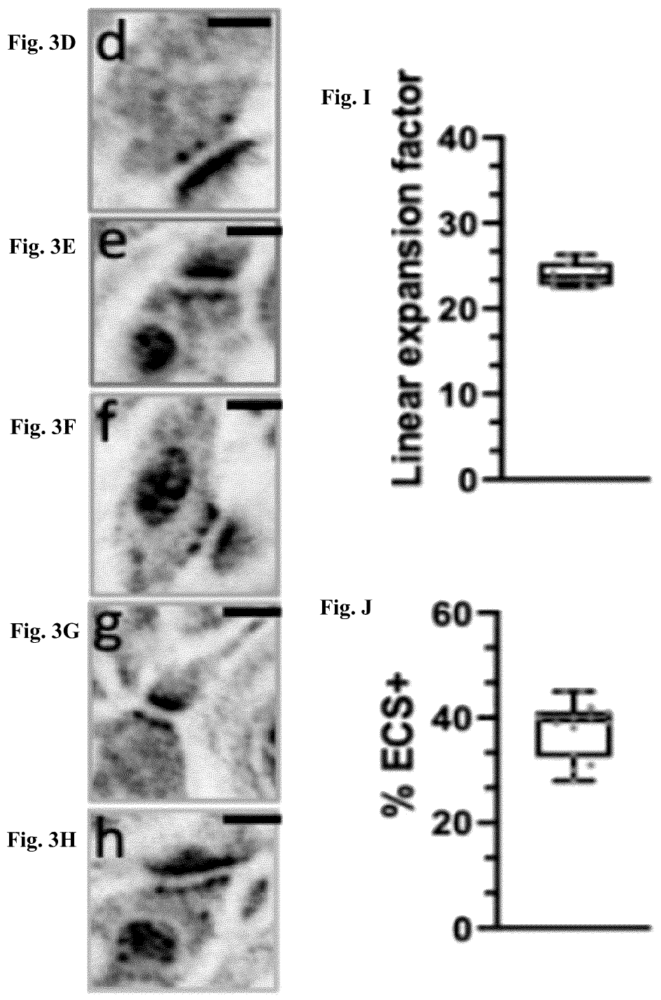

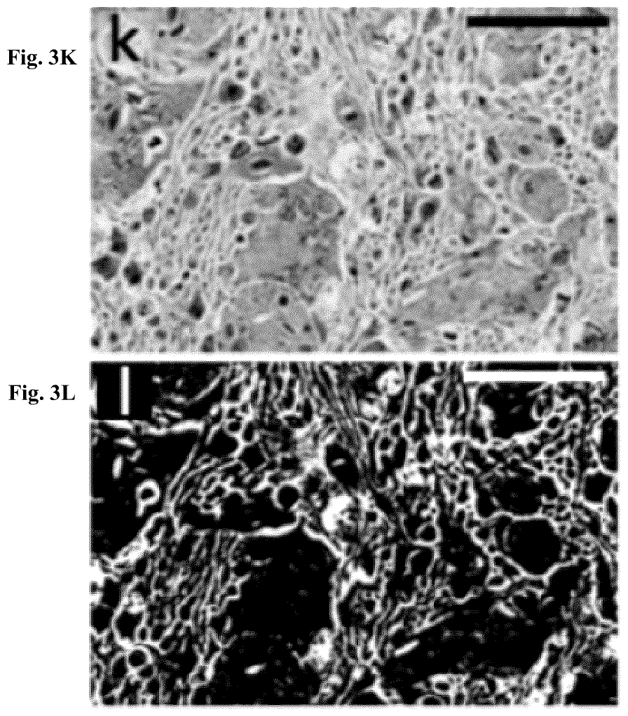

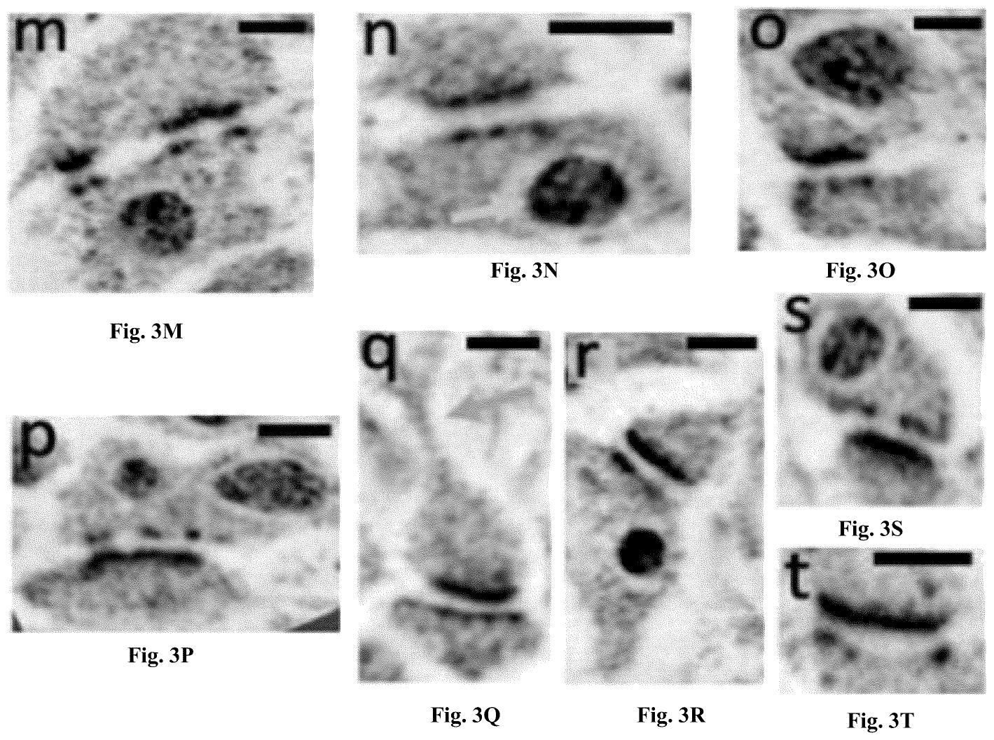

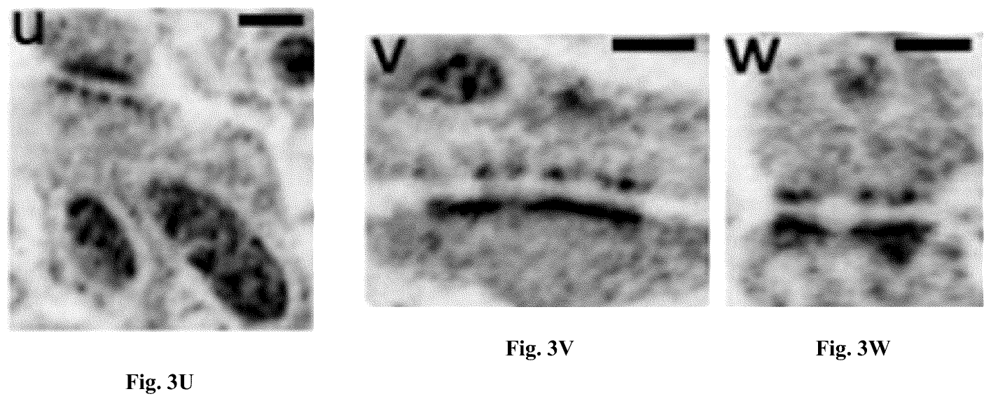

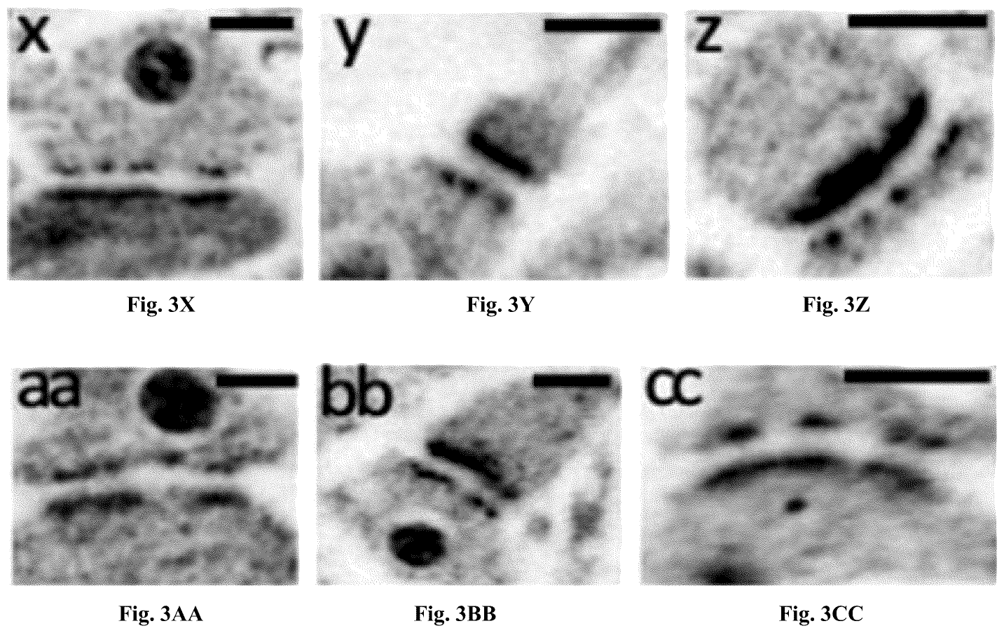

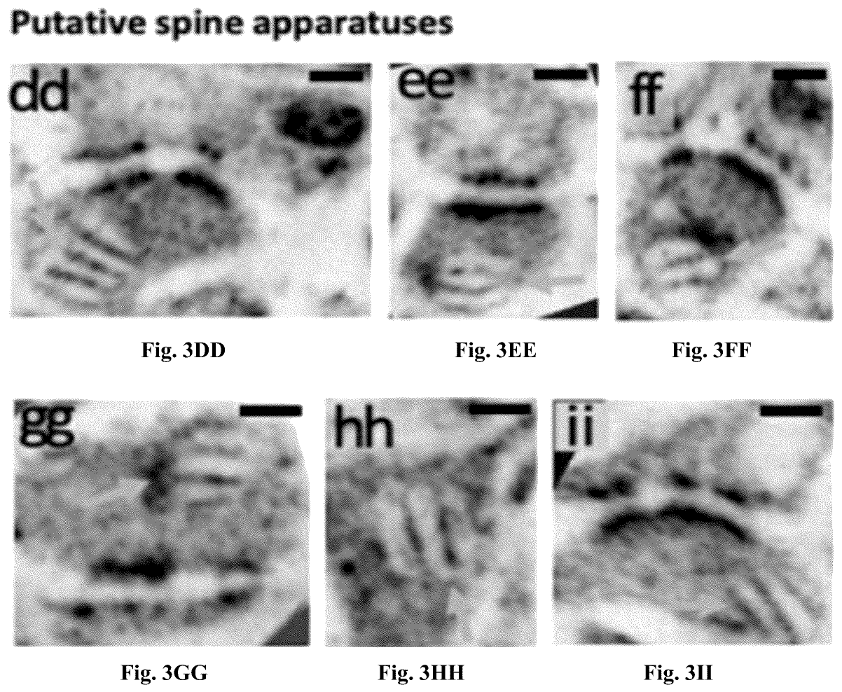

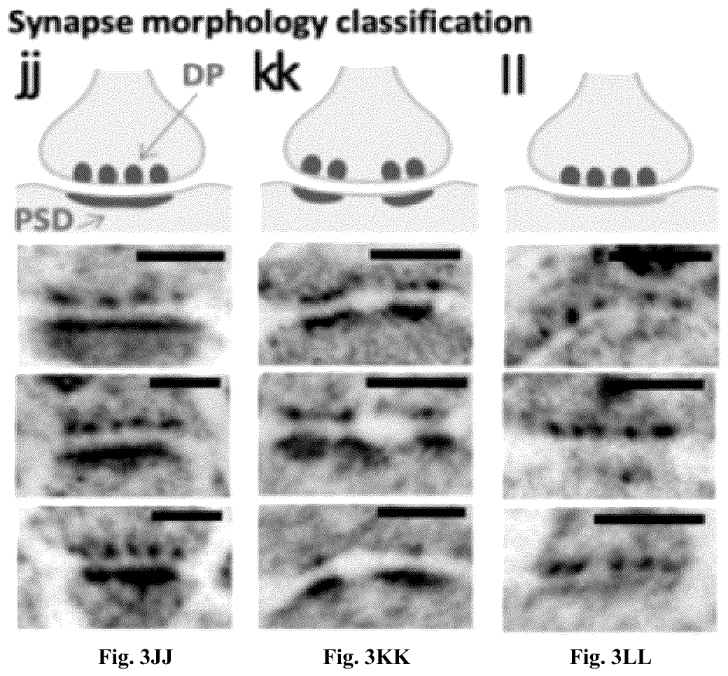

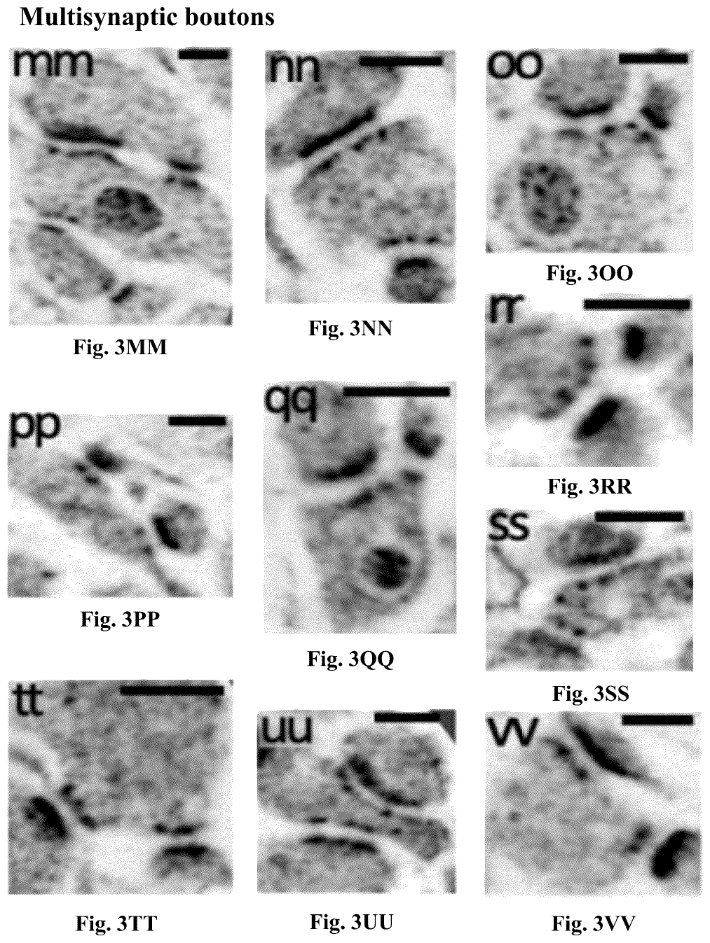

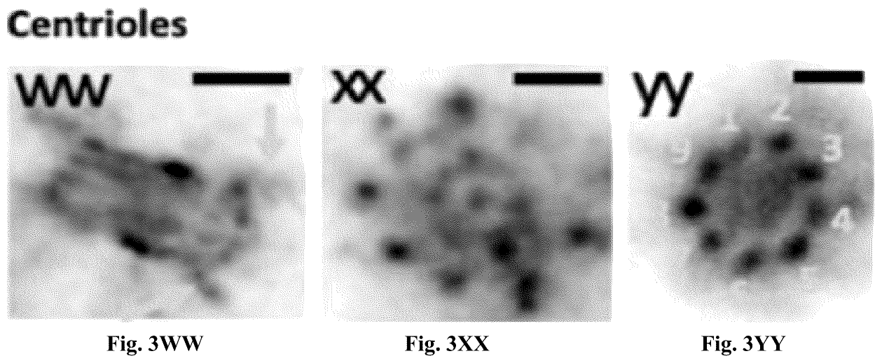

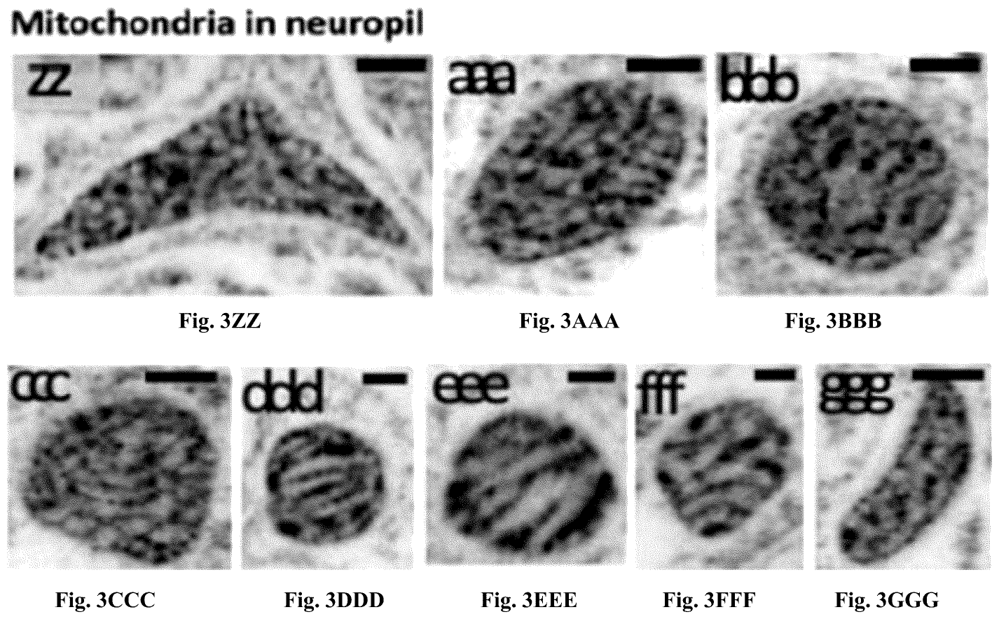

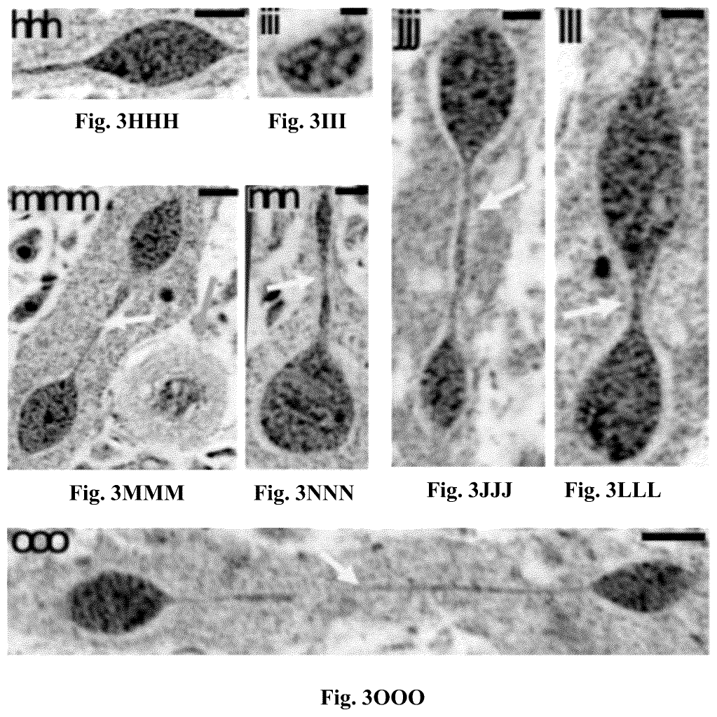

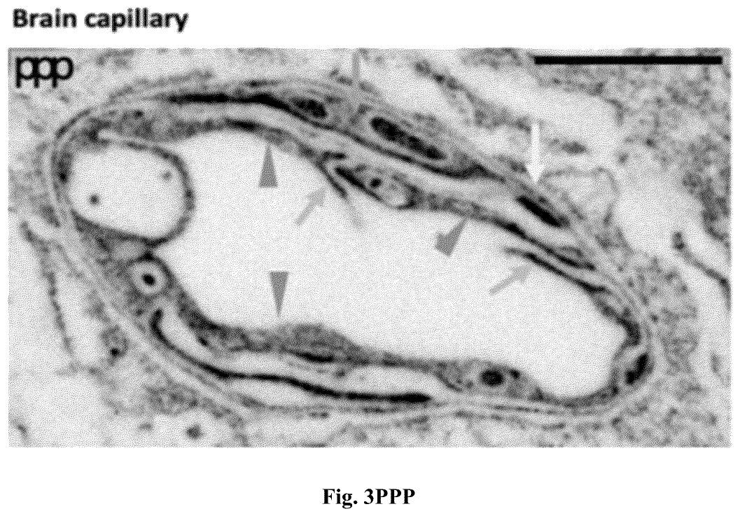

Gels were incubated for 2 h with either 30 μg/mL NHS ester-ATTO594 (Sigma-Aldrich, catalog no. 08741) (ultrastructural samples in FIG. 3) or 30 μg/mL NHS ester-ATTO532 (Sigma-Aldrich, catalog no. 88793) (all immunolabeled and lipid-stained samples), dissolved in 100 mM sodium bicarbonate solution on a rocking platform at RT. The gels were subsequently washed four to six times in either 1×PBS or PBS-T for 20 min each on a rocking platform at RT.

Palmitate pan-staining of neurons

Live and dissociated 80%-confluent hippocampal neurons were incubated with 50 μM azide-functionalized palmitate (Thermofisher, catalog no. C10265) diluted in delipidated medium (DMEM+10% charcoal-stripped FBS; Thermofisher, catalog no. A3382101) for 5 h at 37° C. and 5% CO2. Next, the neurons were fixed with 3% FA+0.1% GA in 1×PBS for 15 min at RT and processed according to the pan-ExM protocol. Prior to NHS ester pan-staining with ATTO532, CuAAC (Copper(I)-catalyzed Azide-Alkyne Cycloaddition) was performed using the CLICK-IT Cell Reaction Buffer Kit (Thermo Fisher, catalog no. C10269) according to manufacturer instructions. Alkyne-functionalized ATTO590 dye (Sigma-Aldrich, catalog no. 93990) was used at a concentration of 5 μM. After CuAAC, the gels were washed twice with 2% (w/v) delipidated BSA (Sigma Aldrich, catalog no. A4612) in 1×PBS for 20 min each on a rocking platform at RT and twice with 1×PBS for 20 min each at RT.

SYTOX Green staining post-expansion

pan-ExM and pan-ExM-t processed gels were incubated with SYTOX Green (Invitrogen, catalog no. 57020) diluted 1:5,000 (dissociated neuron samples) and 1:3,000 (brain tissue samples) in 1×PBS for 30 min on a rocking platform at RT. The gels were then washed three times with PBS-T for 20 min each on a rocking platform at RT.

pan-ExM and pan-ExM-t sample mounting

After expansion, the gels were mounted on glass-bottom dishes (35 mm; no. 1.5; MatTek). A clean 18-millimeter diameter coverslip (Marienfeld, catalog no. 0117580) was put on top of the gels after draining excess water using Kimwipes. The samples were then sealed with two-component silicone glue (Picodent Twinsil, Picodent, Wipperfürth, Germany). After the silicone mold hardened (typically 15-20 min), the samples were stored in the dark at 4° C. until they were imaged.

Image acquisition

All confocal images were acquired using a Leica SP8 STED 3X equipped with a SUPERK EXTREME EXW-12 (NKT Photonics) pulsed white light laser as an excitation source and a ONEFIVE KATANA-08HP pulsed laser as depletion light source (775-nm wavelength). Images were acquired using either an HC FLUOTAR L 25×/0.95 NA water objective, APO 63×/1.2 NA water objective, or an HC PL APO 86×/1.2 NA water CS2 objective. Application Suite X software (LAS X; Leica Microsystems) was used to control imaging parameters. ATTO532 was imaged with 532-nm excitation. ATTO594 was imaged with 585-nm excitation. CF568 was imaged with 568-nm excitation. ATTO647N was imaged with 647-nm excitation. ATTO488 and SYTOX Green were imaged with 488-nm excitation.

Image processing

Images were visualized, smoothed, gamma-, and contrast-adjusted using FIJI/ImageJ software. STED and confocal images were smoothed for display with a 0.4 to 1.5-pixel sigma Gaussian blur. Minimum and maximum brightness were adjusted linearly for optimal contrast.

All line profiles were extracted from the images using the Plot Profile tool in FIJI/ImageJ. Stitching of tiled images was performed using the Pairwise Stitching tool in FIJI/ImageJ.

3D data was visualized using IMARIS software.

Neuron expansion factor calculation

Images of hippocampal mouse and rat neuron cell nuclei in non-expanded and pan-ExM expanded samples stained with SYTOX Green (1:5,000) were acquired with a LEICA SP8 STED 3X microscope using a HCX PL Fluotar 10×/0.30 dry objective. Average nuclear cross-sectional areas were determined using FIJI/ImageJ software. To calculate the expansion factor, the average nuclear cross-sectional area in pan-ExM samples was divided by the average nuclear cross-sectional area of non-expanded samples. The square root of this ratio represents an estimate of the linear expansion factor. For nuclei cross-section measurements in pan-ExM samples, 38 nuclei were analyzed from 4 independent experiments. For nuclei cross-section measurements in non-expanded samples, 279 nuclei were analyzed from 4 independent experiments.

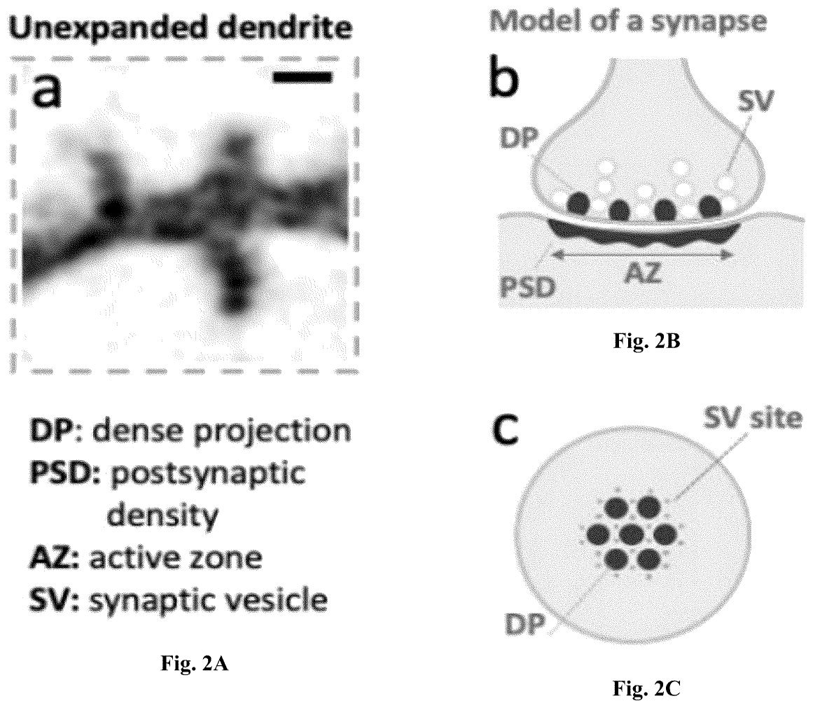

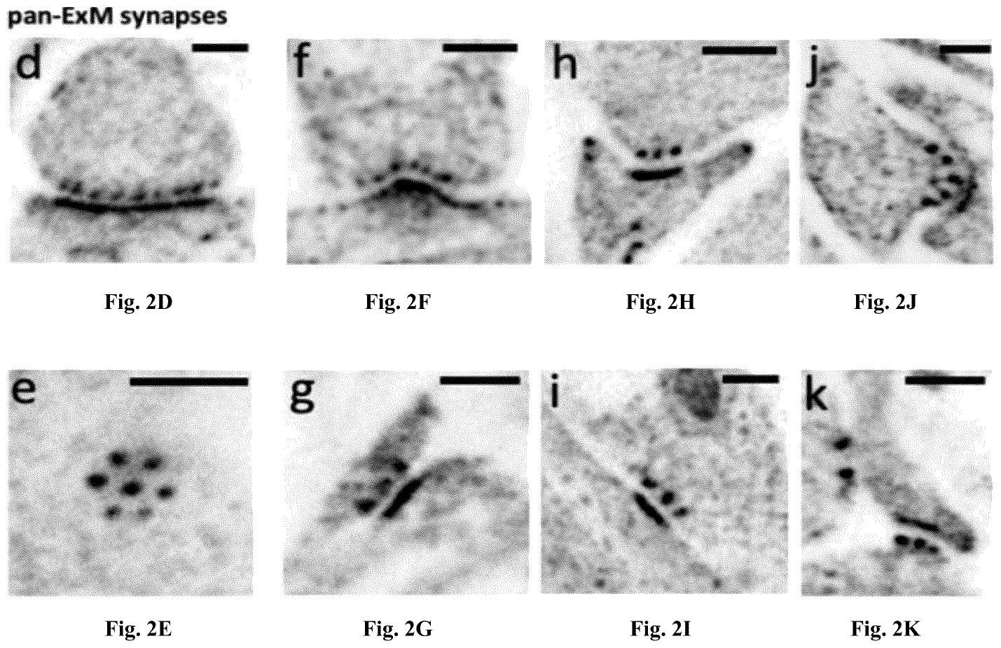

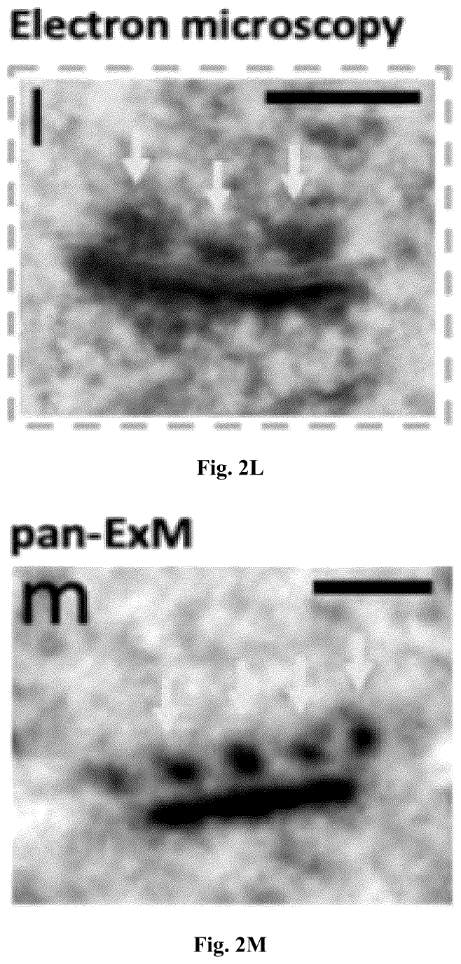

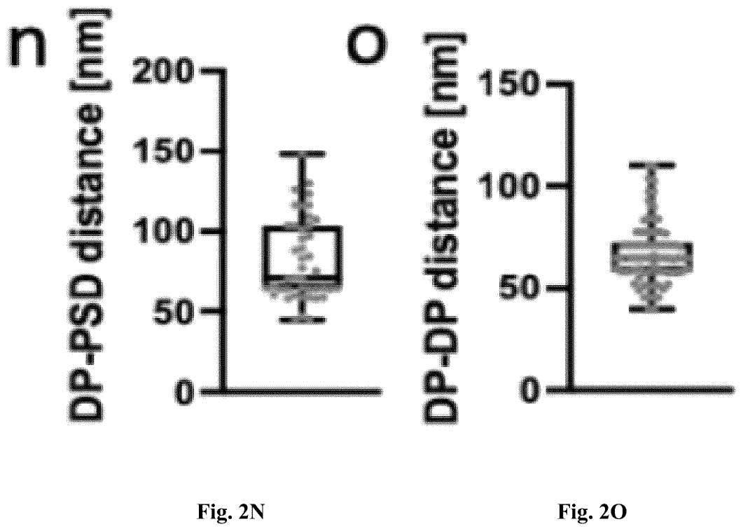

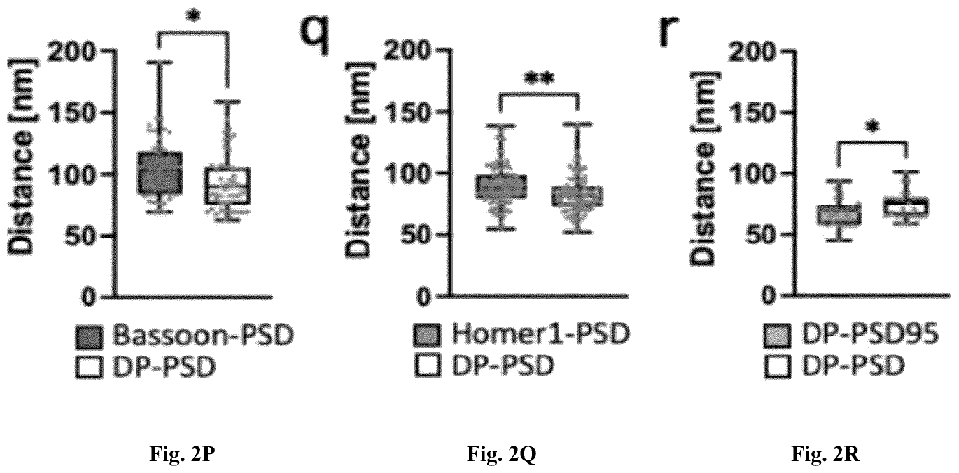

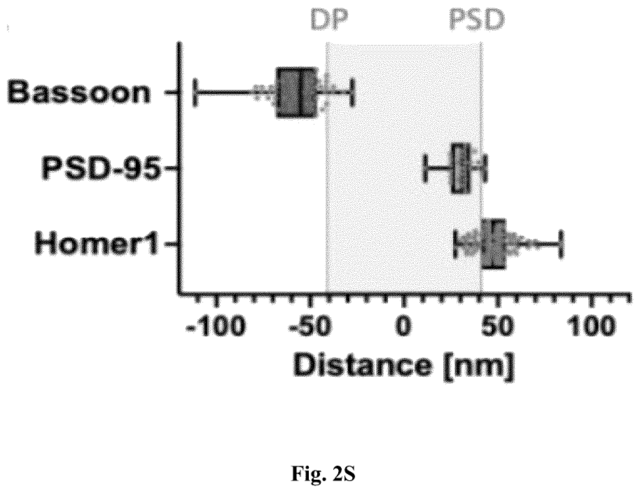

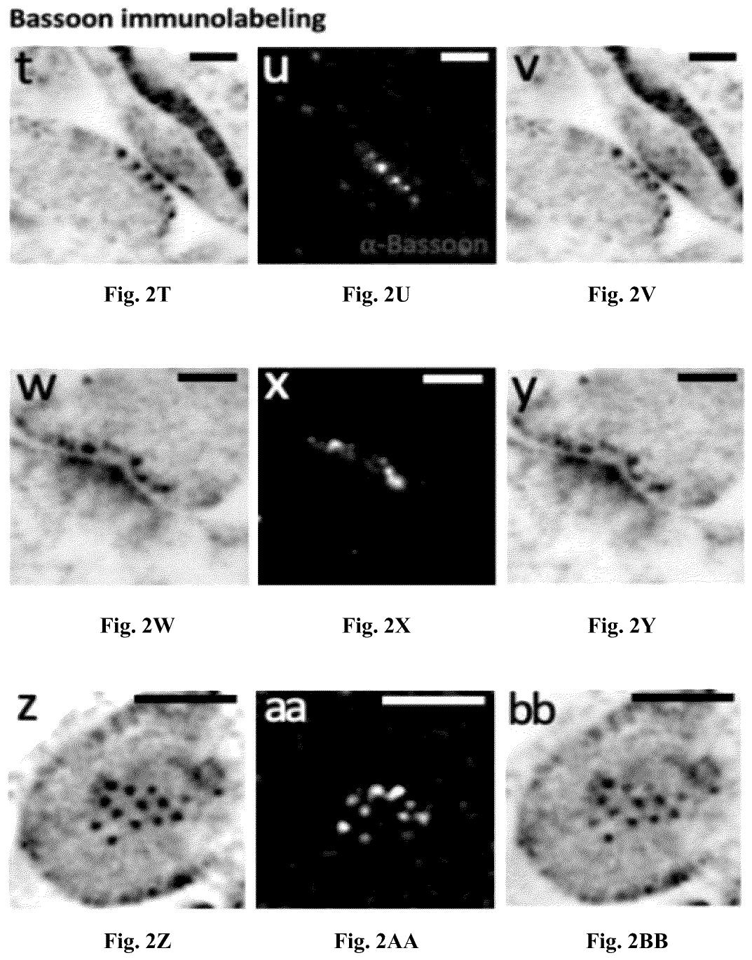

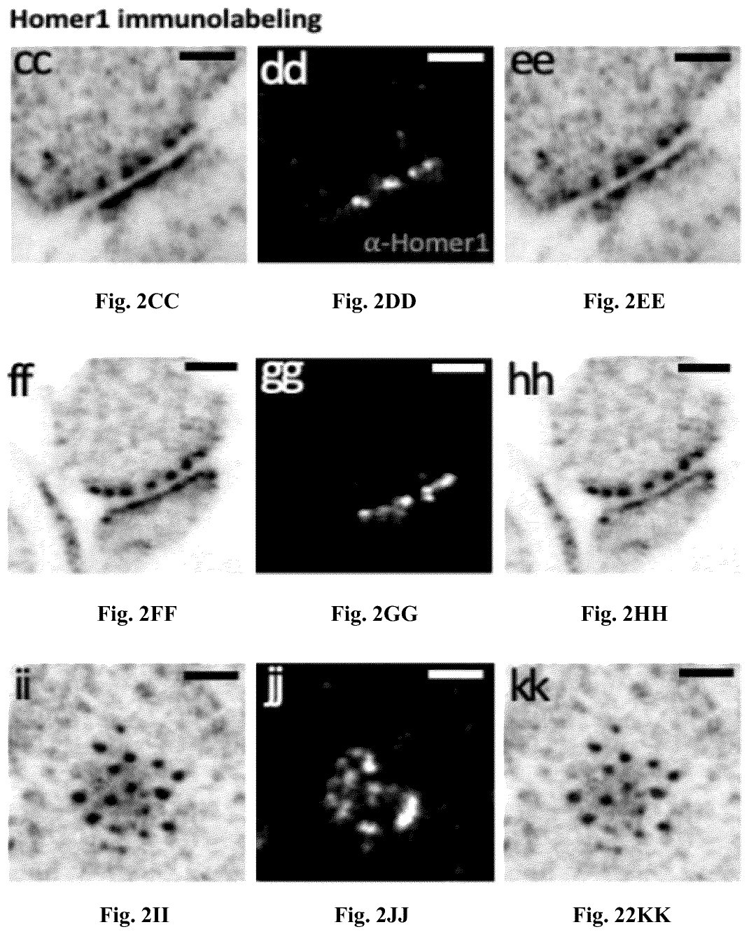

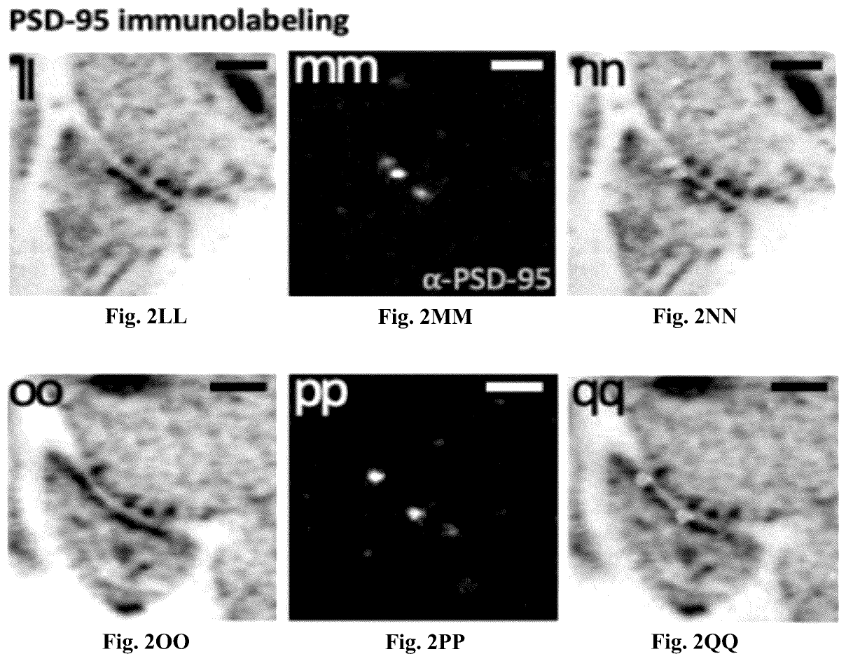

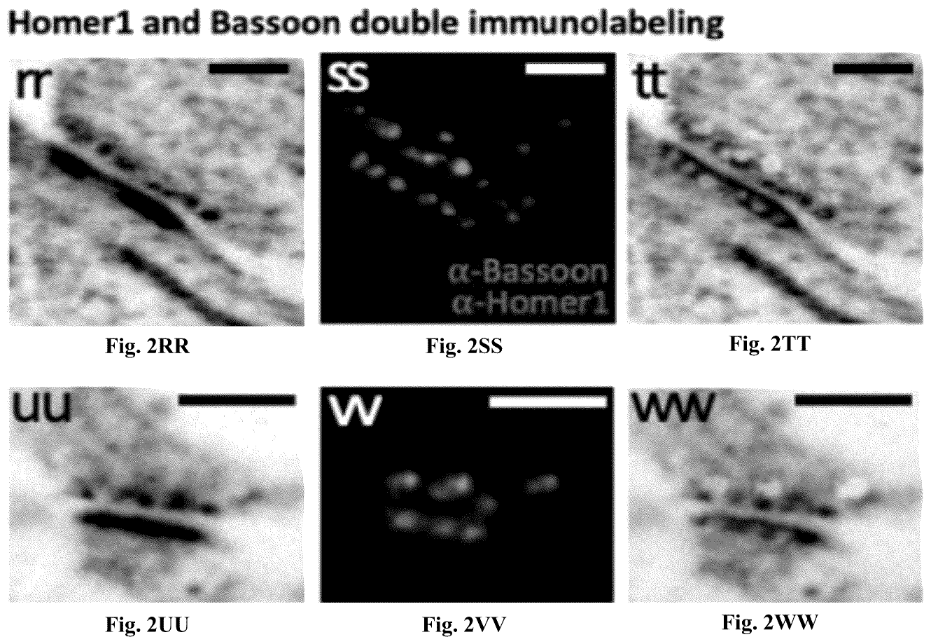

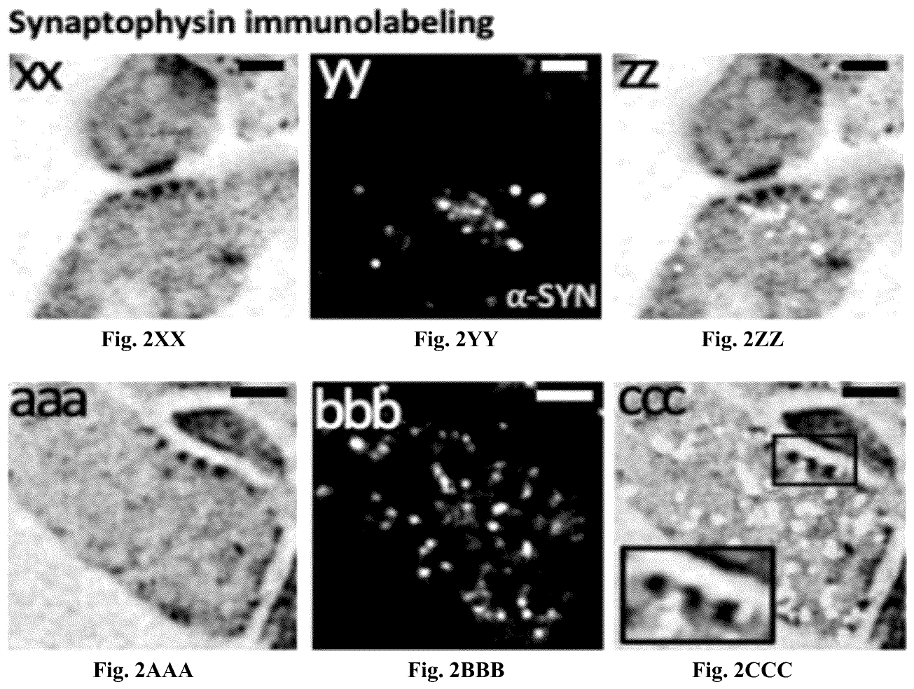

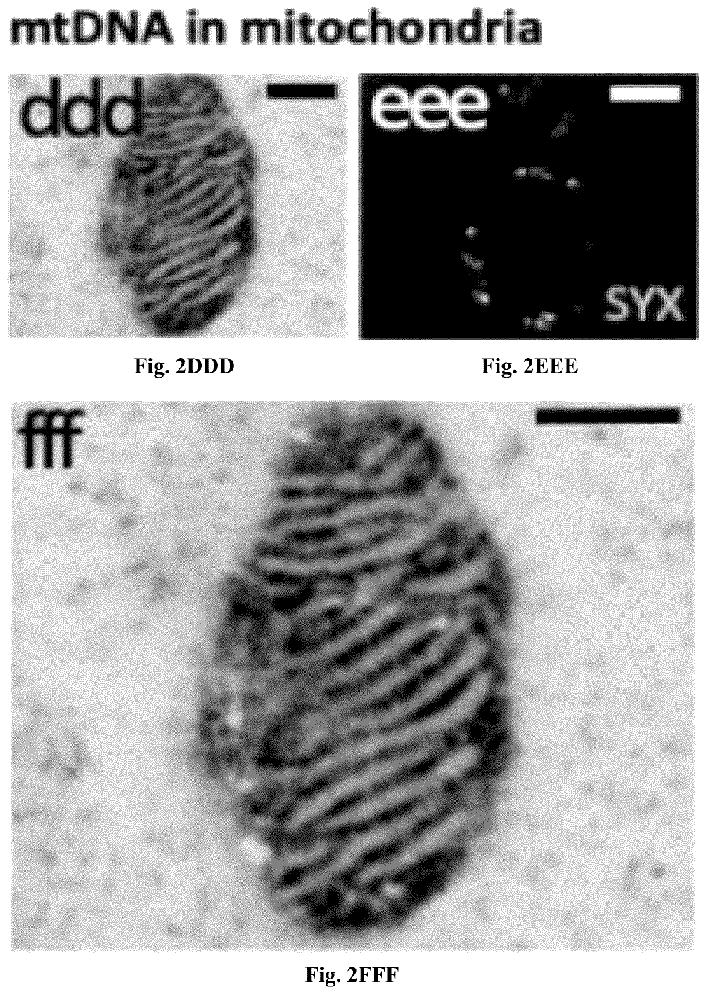

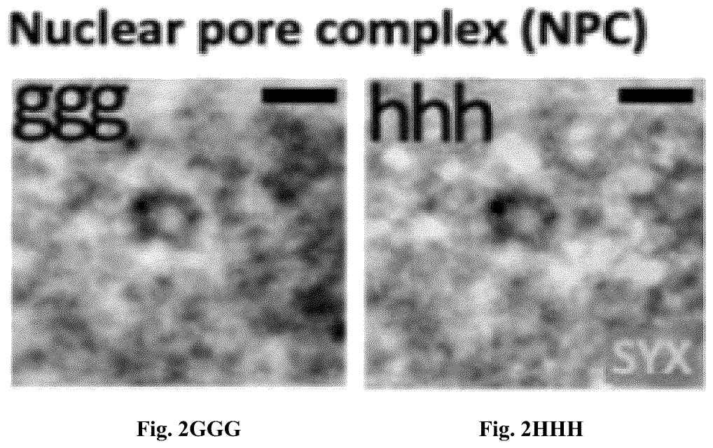

Using 2-pixel thick line profiles in FIJI, the peak-to-peak distances between the intensity distributions of the dense projection (DP) and postsynaptic density (PSD) NHS ester signals in the same samples were measured and divided by the nuclear expansion factor determined above. For this measurement, 44 line profiles were drawn from 4 independent experiments. The DP-PSD value was determined to be 81.9 nm and was used to convert DP-PSD measurements to expansion factors in all subsequent experiments. Results are summarized in FIG. 2n.

General neuron and brain tissue expansion factor calculation

Using 2-pixel thick line profiles in FIJI, the peak-to-peak distances between the intensity distributions of the DP and PSD NHS ester signals were measured and divided by 81.9 nm, the DP-PSD length determined in Neuron expansion factor calculation.

Distance between individual DPs calculation

Using 2-pixel thick line profiles in FIJI, the peak-to-peak distances between the intensity distributions of individual dense projections (DPs) were measured and divided by the determined expansion factor. For this measurement, 78 line profiles were drawn from 41 synapses in 6 independent experiments. Results are summarized in FIG. 2o.

Measurements of synaptic protein distributions in neurons

Using 2-pixel thick line profiles in FIJI, the peak-to-peak distances between the intensity distributions between Homer1 and PSD, PSD-95 and DP, and Bassoon and PSD were measured and divided by the determined experiment expansion factor. For Bassoon-PSD measurements in neurons, 50 line profiles were drawn from 3 independent samples. For Homer1-DP measurements in neurons, 85 line profiles were drawn from 4 independent samples. For PSD95-DP measurements in neurons, 25 line profiles were drawn from 2 independent samples. Results are summarized in FIG. 2p-s.

Measurement of anti-Homer1 signal across dilution factors

Neuron samples were labeled with anti-Homer1 primary antibody and ATTO594-conjugated anti-rabbit antibodies as described in Antibody labeling of neurons post-expansion with this modification: Samples were labeled with primary and secondary antibodies both diluted 1:250 or 1:500 or 1:1000.

Relative protein retention was measured by comparing the peak intensity of Homer1 ATTO504 signal from 2-pixel thick line profiles in FIJI drawn across the punctate signal. For 1:250 measurements, 45 peak intensities were recorded from 7 FOVs. For 1:500 measurements, 13 peak intensities were recorded from 3 FOVs. For 1:1000 measurements, 6 peak intensity measurements were recorded from 1 FOV.

Assessment of fixation effects in brain tissue ultrastructural preservation

Fix-1 brain tissue samples were previously fixed with 4% FA in 1×PBS overnight at 4° C. and sectioned to 70 μm as described in the Brain perfusion section. Next, they were washed three times with 1×PBS for 30 min each on a rocking platform at RT and processed with the pan-ExM-t protocol. Fix-2 brain tissue samples were previously fixed with 4% FA in 1×x PBS overnight at 4° C. and sectioned to 70 μm as described in the Brain perfusion section. Next, they were post-fixed in 0.7% FA+1% AAm in 1×PBS for 7 h at 37° C., washed three times with 1×PBS for 30 min each on a rocking platform at RT, and processed with the pan-ExM-t protocol. Fix-3 brain tissue samples were previously fixed with 4% FA in 1×PBS overnight at 4° C. and sectioned to 70 μm as described in the Brain perfusion section. Next, they were post-fixed in 4% FA+20% AAm in 1×PBS for 7 h at 37° C., washed three times with 1×PBS for 30 min each on a rocking platform at RT, and processed with the pan-ExM-t protocol. Fix-4 brain tissue samples were previously fixed with 4% FA+0.1% GA in 1×PBS overnight at 4° C. and sectioned to 70 μm as described in the Brain perfusion section. Next, they were post-fixed in 4% FA+20% AAm in 1×PBS for 12 h at 4° C., washed three times with 1×PBS for 30 min each on a rocking platform at RT, and processed with the pan-ExM-t protocol. Fix-5 brain tissue samples were previously fixed with 4% FA in 1×PBS overnight at 4° C. and sectioned to 70 μm as described in the Brain perfusion section. Next, they were treated with 0.1 mg/mL acryloyl-X, SE (AcX; Thermofisher, catalog no. A20770) in 1×PBS for 3 h at RT, washed three times with 1×PBS for 30 min each on a rocking platform at RT, and processed with the pan-ExM-t protocol. Fix-6 brain tissue was previously fixed with 4% FA+20% AAm in 1×PBS overnight at 4° C. and sectioned to 70 μm as described in the Brain perfusion section. Next, they were washed three times with 1×PBS for 30 min each on a rocking platform at 4° C. and processed with the pan-ExM-t protocol.

Expansion factors were calculated as described in the General neuron and brain tissue expansion factor calculation section. For Fix-2 DP-PSD measurements, 38 line profiles were drawn from 6 FOVs. For Fix-3 DP-PSD measurements, 81 line profiles were drawn from 9 FOVs. For Fix-4 DP-PSD measurements, 77 line profiles were drawn from 8 FOVs. For Fix-5 DP-PSD measurements, 74 line profiles were drawn from 9 FOVs. For Fix-6 DP-PSD measurements, 254 line profiles were drawn from 10 FOVs in 3 independent experiments.

To calculate the ECS+lipid membrane (ECS+) fraction, ˜250×250 μm2 large-FOV images that show predominantly neuropil pan-stained with NHS ester, were processed with a Gaussian blur of 1 sigma (Fix-1, Fix-2 and Fix-5) or 0.5 sigma (Fix-3, Fix-4 and Fix-6) in Fiji. A mask created with manual thresholding was used to exclude large gaps in the image that do not represent neurites. The images were then thresholded using the Otsu method (Otsu, 1979) with 100%, 70%, 50%, and 30% of the automatically determined threshold. The ECS+fraction was calculated by dividing the total thresholded pixels by the total area represented by the masked pixels. For Fix-2, 8 FOVs from 1 independent experiment were processed. For Fix-3, 8 FOVs from 1 independent experiment were processed. For Fix-4, 8 FOVs from 1 independent experiment were processed. For Fix-5, 8 FOVs from 1 independent experiment were processed. For Fix-6, 14 FOVs from 3 independent experiments were processed.

Assessment of denaturation effects in brain tissue expansion factor

Denat-4, Denat-6, and Denat-8 brain tissue samples were previously fixed with 4% FA+20% AAm in 1×PBS overnight at 4° C. and sectioned to 70 μm as described in the Brain perfusion section. Next, they were washed three times with 1×PBS for 30 min each on a rocking platform at RT and processed with the pan-ExM-t protocol with these modifications: Denat-6 samples were denatured for 6 h and Denat-8 samples were denatured for 8 h.

Expansion factors were calculated as described in the General neuron and brain tissue expansion factor calculation section. For Denat-4 DP-PSD measurements, 50 line profiles were drawn from 3 FOVs. For Denat-6 DP-PSD measurements, 42 line profiles were drawn from 3 FOVs. For Denat-8 DP-PSD measurements, 100 line profiles were drawn from 3 FOVs.

Relative protein retention was measured by comparing the peak intensity of DP NHS ester signal from 2-pixel thick line profiles in FIJI. For Denat-4 measurements, 50 DP peak intensities were recorded from 3 FOVs. For Denat-6 measurements, 42 DP peak intensities were recorded from 3 FOVs. For Denat-8 measurements, 48 DP peak intensity measurements were recorded from 3 FOVs. The intensity values were multiplied by the cube of the expansion factor determined for every denaturation condition.

Assessment of SA monomer concentration effects in brain tissue preservation

19SA/19SA, 19SA/9SA, 9SA/19SA, and 9SA/9SA brain tissue samples were previously fixed with 4% FA+20% AAm in 1×PBS overnight at 4° C. and sectioned to 70 μm as described in the Brain perfusion section. Next, they were washed three times with 1×PBS for 30 min each on a rocking platform at RT and processed with the pan-ExM-t protocol with these modifications: the monomer composition of the second expansion hydrogel in 19SA/9SA samples was 10% (w/v) AAm+9% SA (w/v); the monomer composition of the first expansion hydrogel in 9SA/19SA samples was 10% (w/v) AAm+9% SA (w/v); and the monomer composition of the first and second expansion hydrogels in 9SA/9SA samples was 10% (w/v) AAm+9% SA (w/v).

Expansion factors were calculated as described in the General neuron and brain tissue expansion factor calculation section. For 19SA/19SA DP-PSD measurements, 254 line profiles were drawn from 10 FOVs in 3 independent experiments. For 19SA/9SA DP-PSD measurements, 267 line profiles were drawn from 12 FOVs in 2 independent experiments. For 9SA/19SA DP-PSD measurements, 88 line profiles were drawn from 6 FOVs in 2 independent experiments. For 9SA/9SA DP-PSD measurements, 86 line profiles were drawn from 6 FOVs in 2 independent experiments.

Measurements of synaptic protein distributions in brain tissue

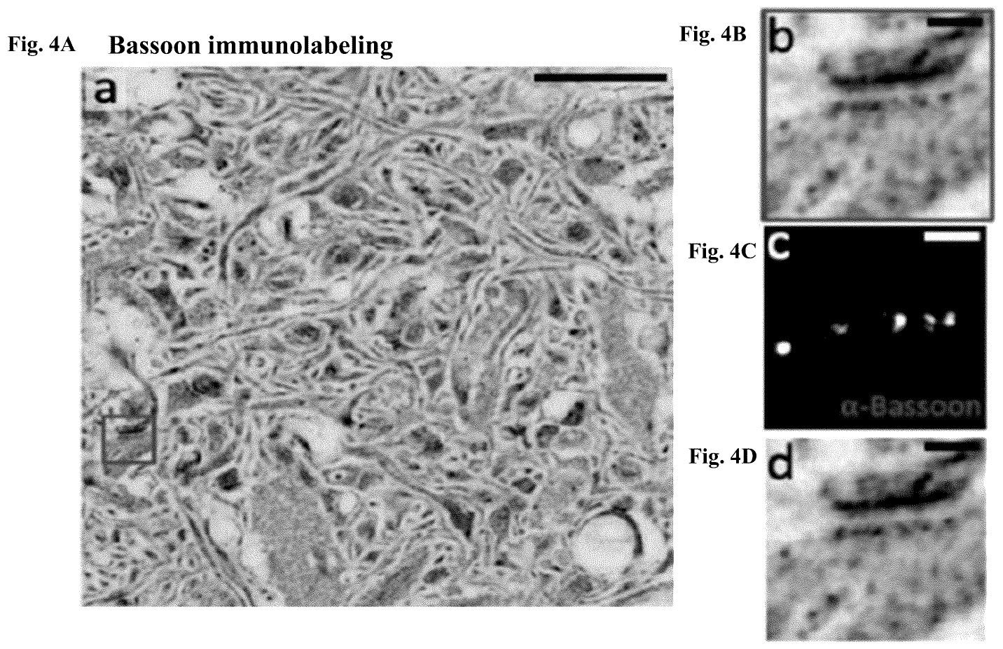

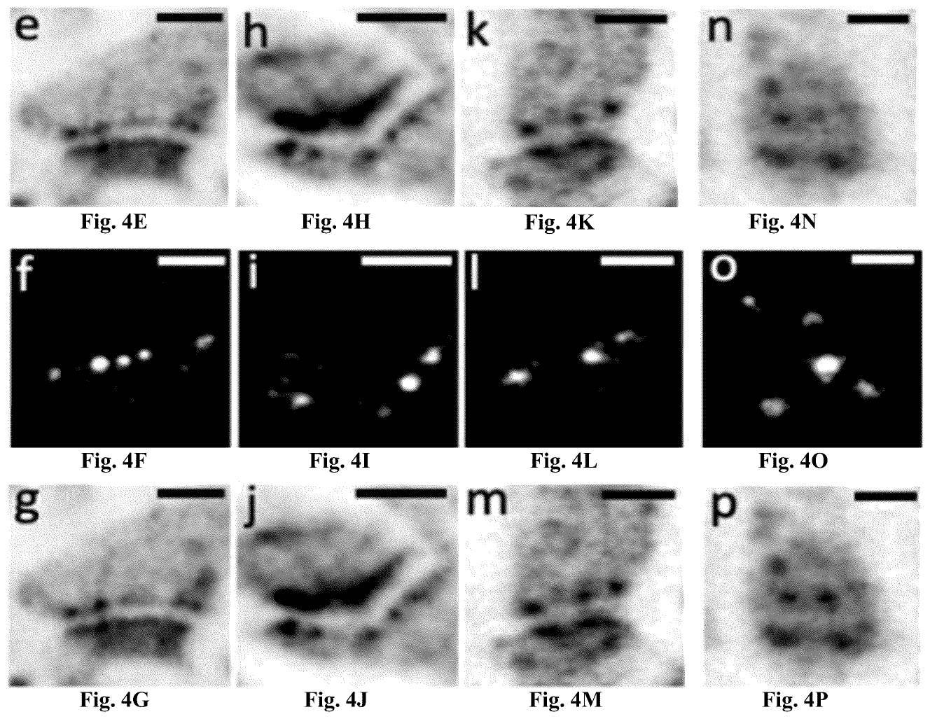

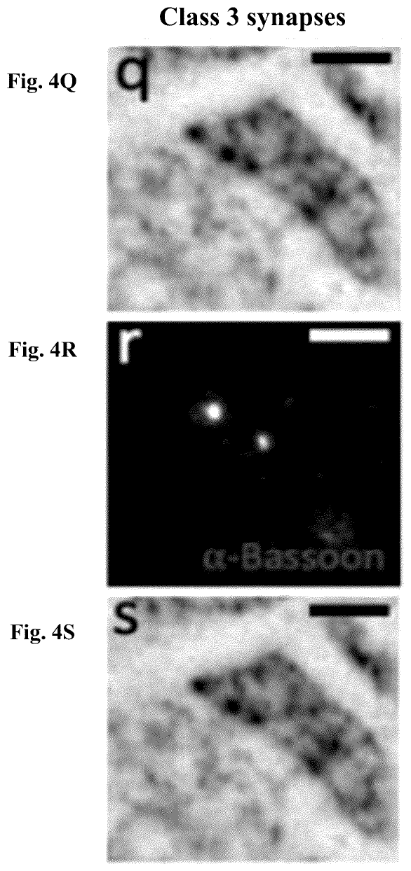

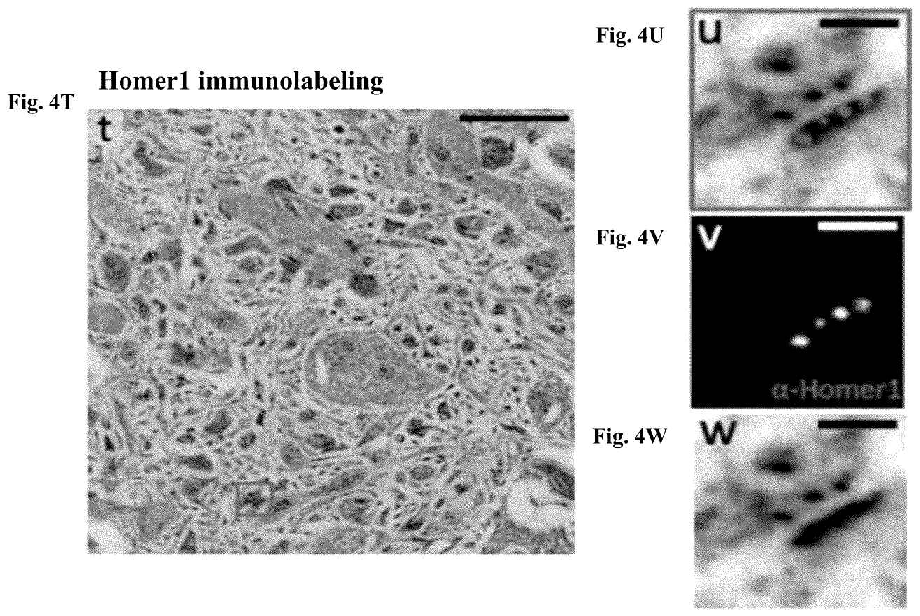

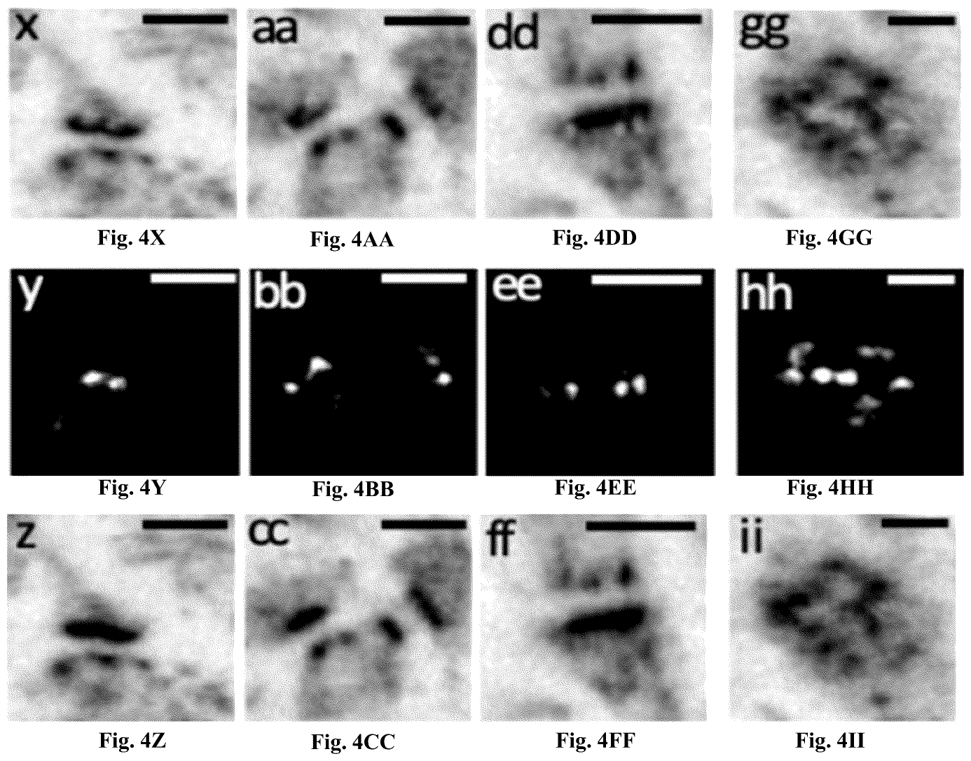

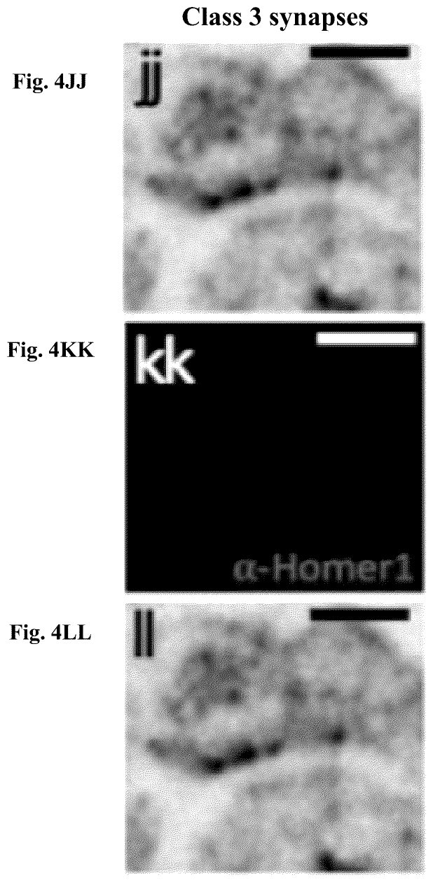

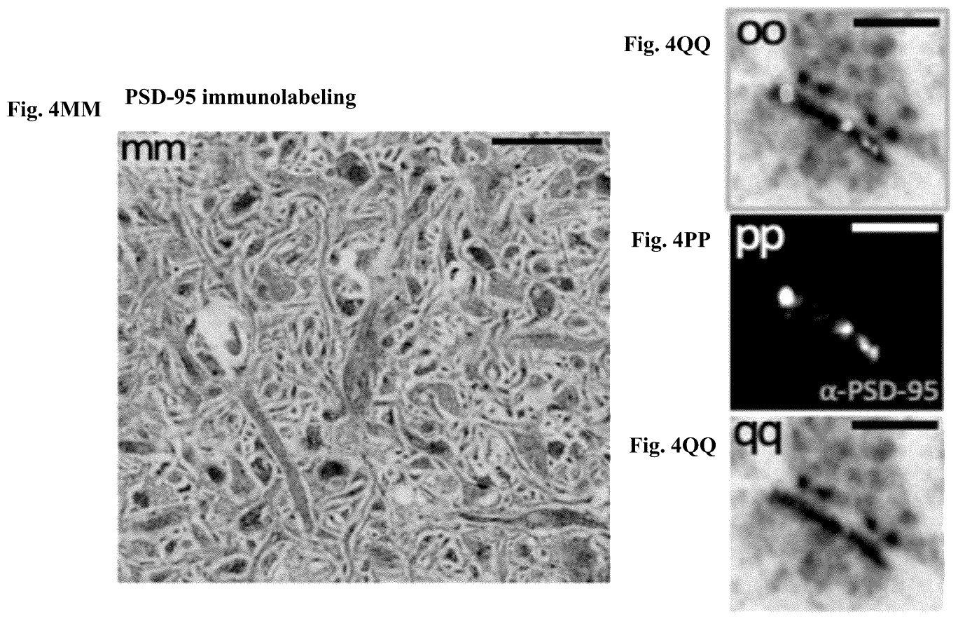

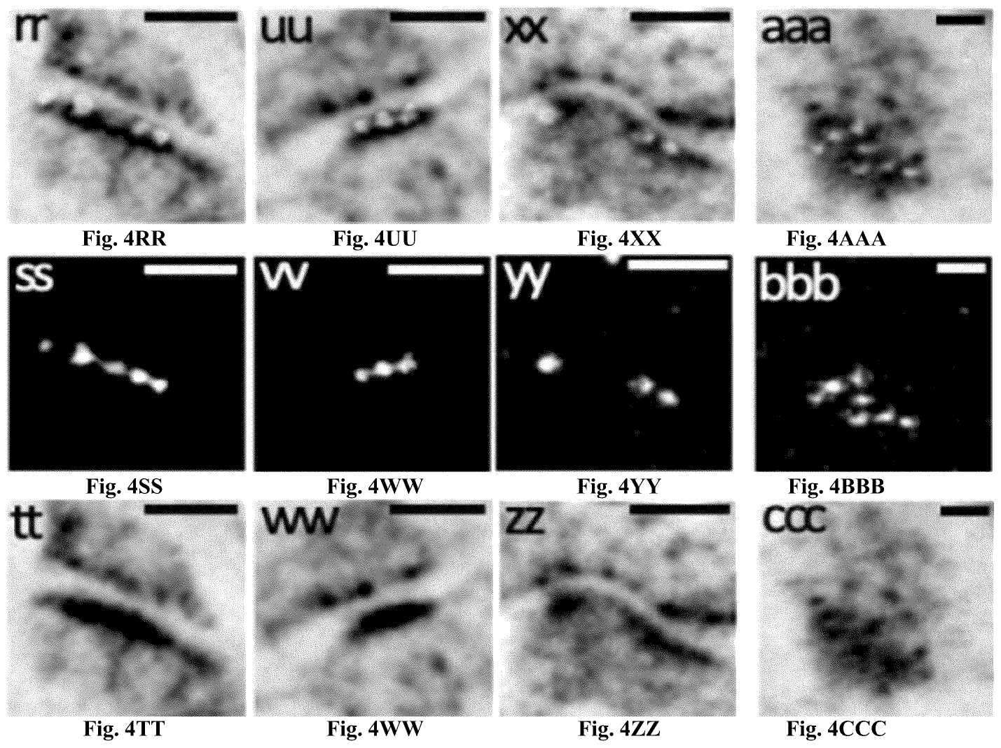

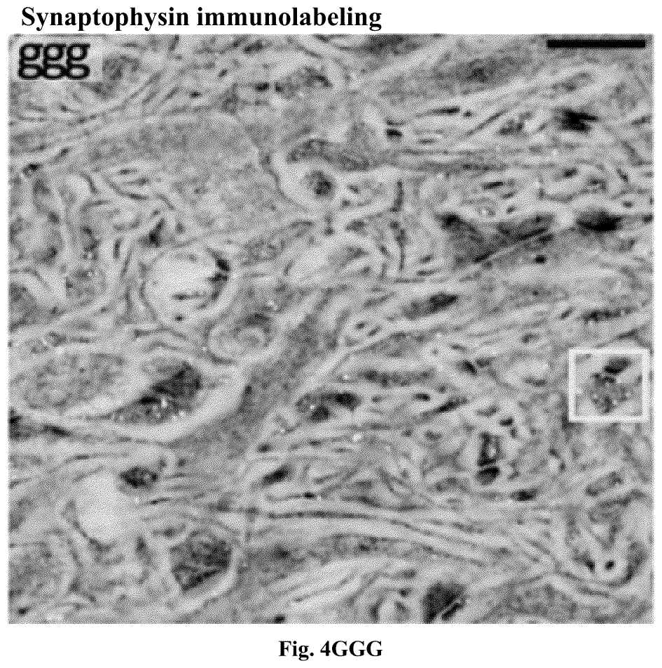

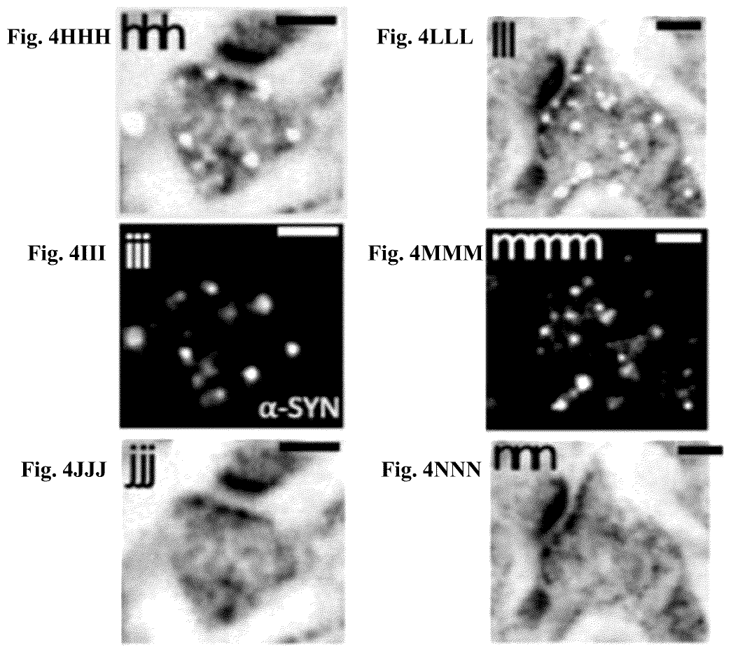

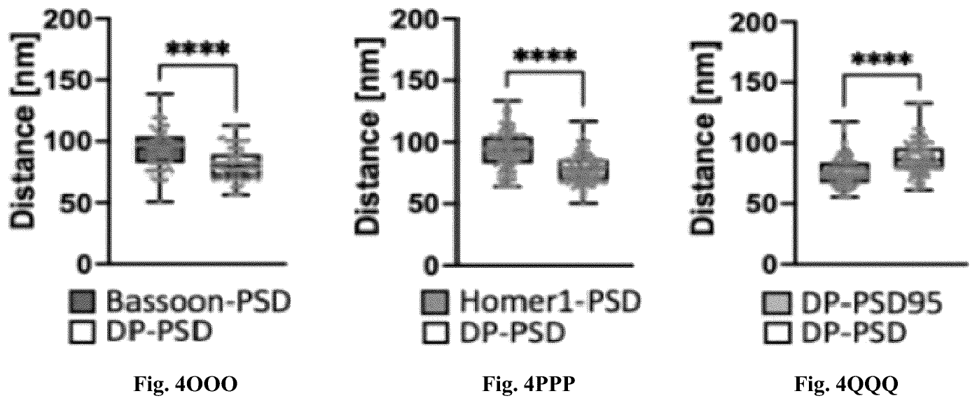

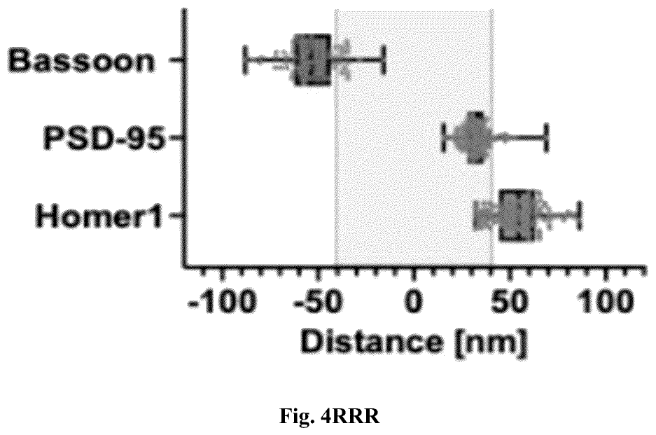

Using 2-pixel thick line profiles in FIJI, the peak-to-peak distances between the intensity distributions between Homer1 and PSD, PSD-95 and DP, and Bassoon and PSD were measured and divided by the determined experiment expansion factor. For Homer1-DP measurements in brain tissue, 120 line profiles were drawn from 5 FOVs in 2 independent experiments. For PSD-95-DP measurements in brain tissue, 113 line profiles were drawn from 3 FOVs in one independent experiment. For Bassoon-PSD measurements in brain tissue, 74 line profiles were drawn from 3 FOVs in one independent experiment. Results are summarized in FIG. 4, Panels (ooo-rrr).

Measurement of BDP signal in 4-fold expanded brain tissue across fixation conditions

To compare the effect of fixation on lipid retention, brain tissue was fixed with either 4% FA in 1×PBS (FA-fix) or 4% FA+0.1% GA in 1×PBS (FA/GA-fix) overnight at 4° C. and sectioned to 70 μm as described in the Brain perfusion section. Several FA/GA-fix tissue sections were post-fixed with 0.01% osmium tetroxide (catalog no.) in 1×PBS for 1 h at RT (FA/GA/OsO4-fix), washed three times with 1×PBS at RT, and stored in 4° C. until subsequent treatment. All tissue sections were post-fixed in 4% FA+20% AAm in 1×PBS for 15 h at 4° C., washed three times with 1×PBS for 30 min each on a rocking platform at 4° C., and processed only with the First round of expansion for brain tissue protocol with this modification: tissue sections were denatured in SDS buffer for 1 h. For NHS ester pan-staining, gels were incubated with 10 μg/mL NHS ester ATTO532 (Sigma-Aldrich, catalog no. 88793) in 1×PBS for 30 min on a rocking platform at RT and washed three times with 1×PBS for 30 min each. For BDP pan-staining, gels were incubated with 10 μM BODIPY TR Methyl Ester (Thermofisher, catalog no. C34556) in 1×PBS for 1 h on a rocking platform at RT and washed three times with 1×PBS for 30 min each. Gels were subsequently expanded in MilliQ water and imaged.

BDP pan-staining signal in axons was measured by recording the peak intensity of BDP signal from 10-pixel thick line profiles drawn across the width of axons in FIJI. For FA-fix measurements, 46 peak intensities were recorded from 5 FOVs. For FA/GA-fix measurements, 46 peak intensities were recorded from 5 FOVs. For FA/GA/OsO4-fix measurements, 38 peak intensities were recorded from 5 FOVs.

Measurement of BDP signal in 4-fold expanded brain tissue across denaturation conditions

To compare the effect of fixation on lipid retention, brain tissue was fixed with 4% FA+0.1% GA in 1×PBS overnight at 4° C. and sectioned to 70 μm as described in the Brain perfusion section. All tissue sections were post-fixed in 4% FA+20% AAm in 1×PBS for 15 h at 4° C., washed three times with 1×PBS for 30 min each on a rocking platform at 4° C., and processed only with the First round of expansion for brain tissue protocol with this modification: tissue sections were denatured with either SDS buffer at 75° C. for 1 h (SDS-1h), or SDS buffer at 75° C. for 4 h, (SDS-4h), non-denatured (PBS), or denatured with guanidine hydrochloride (G-HCl) buffer (6 M G-HCl+5 mM DTT+50 mM Tris in MilliQ water, pH 7.5) at 42° C. overnight. For NHS ester pan-staining, gels were incubated with 10 μg/mL NHS ester ATTO532 (Sigma-Aldrich, catalog no. 88793) in 1×PBS for 30 min on a rocking platform at RT and washed three times with 1×PBS for 30 min each. For BDP pan-staining, gels were incubated with 10 μM BODIPY TR Methyl Ester (Thermofisher, catalog no. C34556) in 1×PBS for 1 h on a rocking platform at RT and washed three times with 1×PBS for 30 min each. Gels were subsequently expanded in MilliQ water and imaged.

BDP pan-staining signal in axons was measured by recording the peak intensity of BDP signal from 10-pixel thick line profiles drawn across the width of axons in FIJI. For SDS-1 h measurements, 40 peak intensities were recorded from 4 FOVs. For SDS-4 h measurements, 20 peak intensities were recorded from 1 FOV. For PBS measurements, 41 peak intensities were recorded from 2 FOVs. For G-HCl measurements, 24 peak intensities were recorded from 1 FOV.

BDP pan-staining signal in mitochondria was measured by recording the peak intensity of BDP signal from 10-pixel thick line profiles drawn across the width of mitochondria in FIJI. For SDS-1 h measurements, 40 peak intensities were recorded from 4 FOVs. For SDS-4 h measurements, 18 peak intensities were recorded from 1 FOV. For PBS measurements, 41 peak intensities were recorded from 2 FOVs. For G-HCl measurements, 20 peak intensities were recorded from 1 FOV.

Measurement of pacSph pan-staining signal across UV irradiation conditions

To compare the effect of fixation on lipid retention, brain tissue was fixed with 4% FA+20% AAm in 1×PBS overnight at 4° C. and sectioned to 70 μm as described in the Brain perfusion section. Sections were incubated in 50 μM pacSph (PhotoClick Sphingosine; Avanti Polar Lipids, catalog no. 900600) in 1×PBS overnight at 4° C., washed three times in 1×PBS for 1 h each at 4° C., and either stored at 4° C. (noUV) or photo-crosslinked. Samples that were photo-crosslinked were kept on ice and irradiated with an 8 Watt, 365 nm UV light source (Analytik Jena, UVL-18) 1 cm away from a lamp surface for 30 min on both section surface sides, either before hydrogel embedding (UV-before) or immediately after First round of expansion for brain tissue while the tissue-hydrogel hybrid was still sandwiched between a glass microscope slide and glass coverslip (UV-after). Samples were processed with the remaining steps of the pan-ExM-t protocol with this modification: before NHS ester pan-staining, CuAAC was performed using the Click-iT Protein Reaction Buffer Kit (Thermo Fisher, catalog no. C10276) according to manufacturer instructions. Azide-functionalized ATTO590 dye (ATTO-TEC, catalog no. AD 590-101) was used at a concentration of 5 μM. After CuAAC, the gels were washed once with 2% (w/v) delipidated BSA (Sigma Aldrich, catalog no. A4612) in 1×PBS for 20 min each and three times with 1×PBS for 20 min each on a rocking platform at RT.

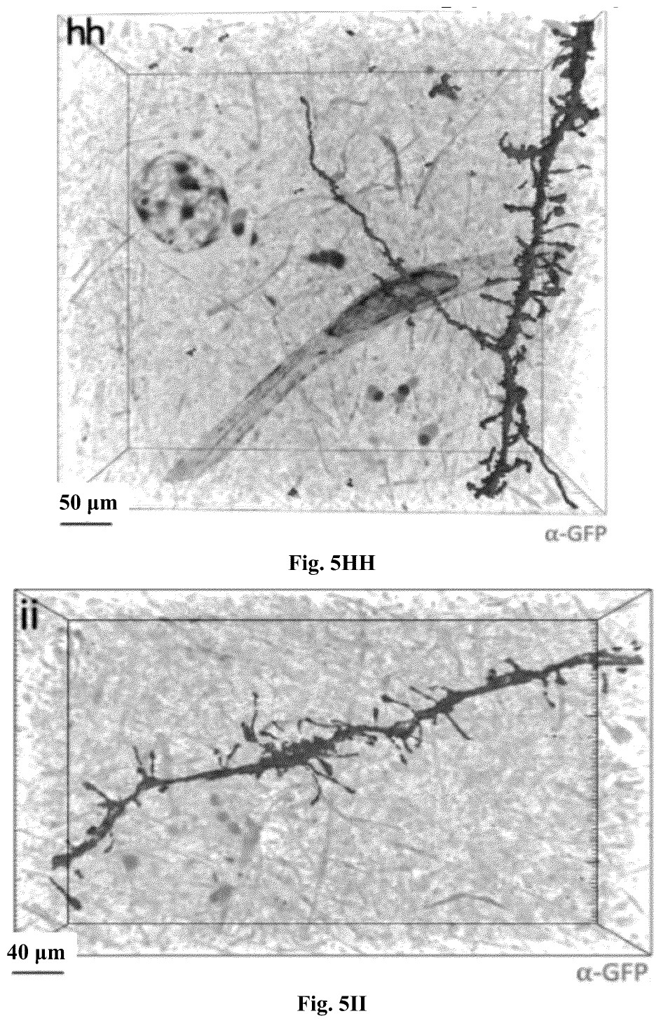

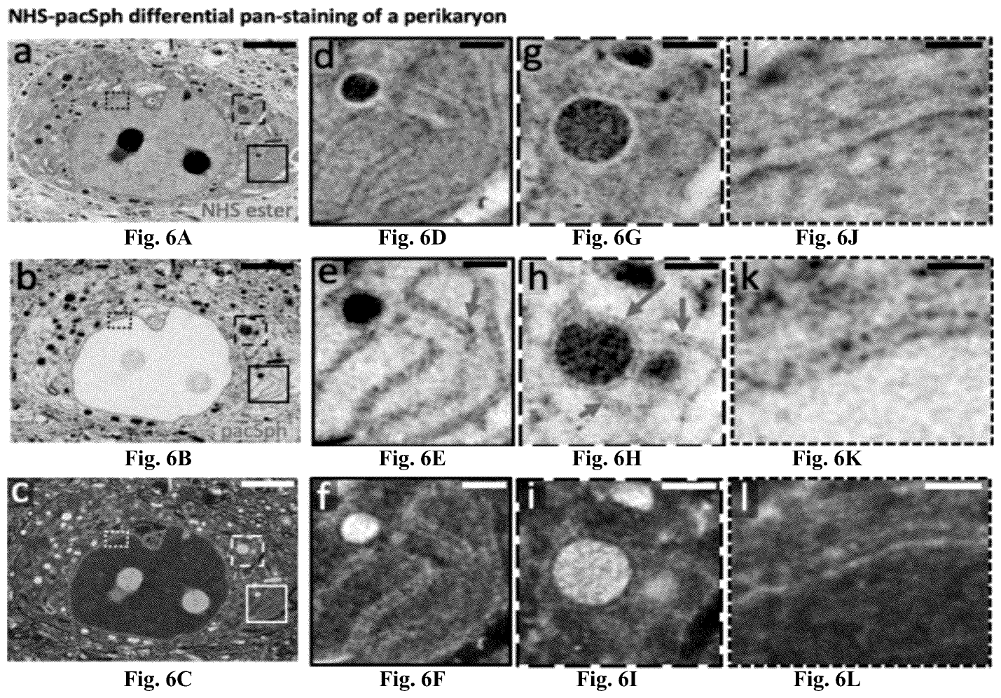

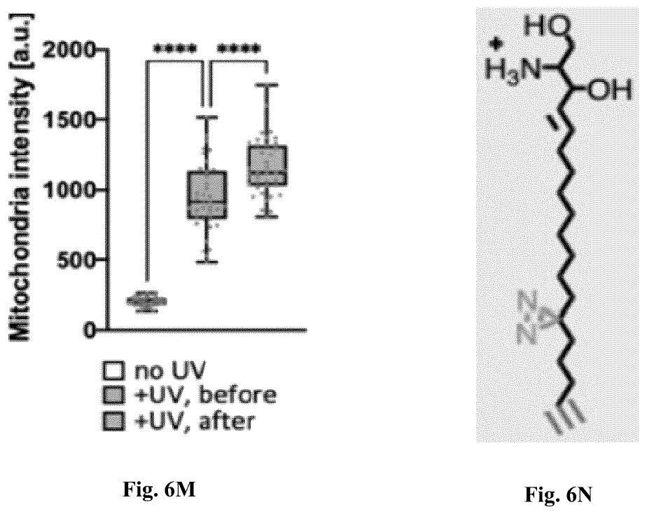

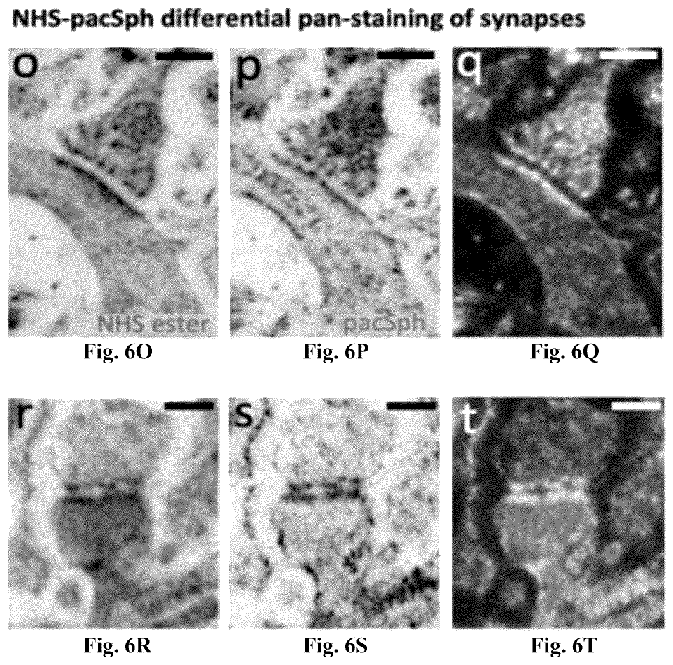

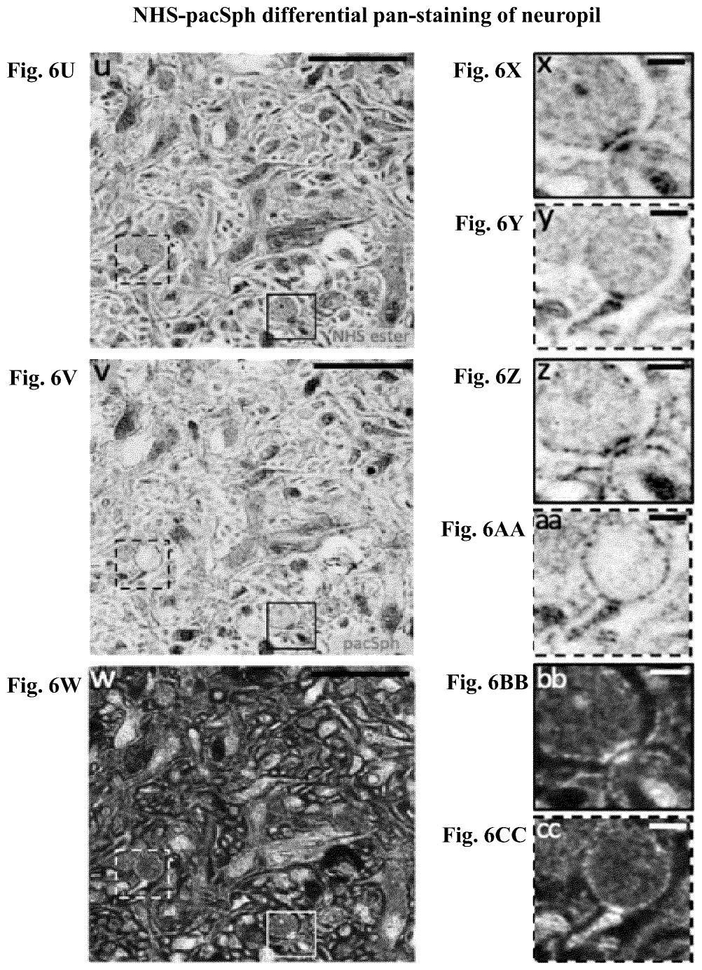

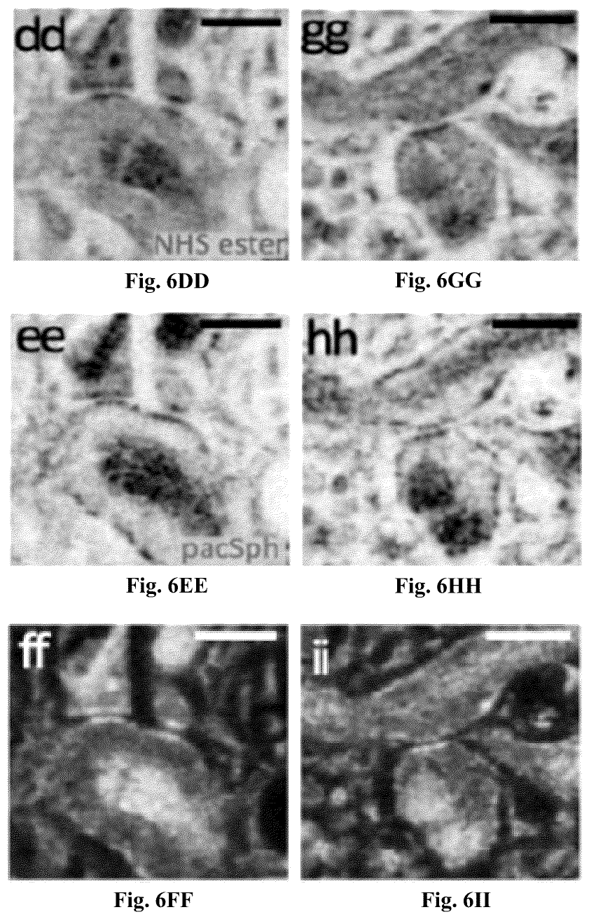

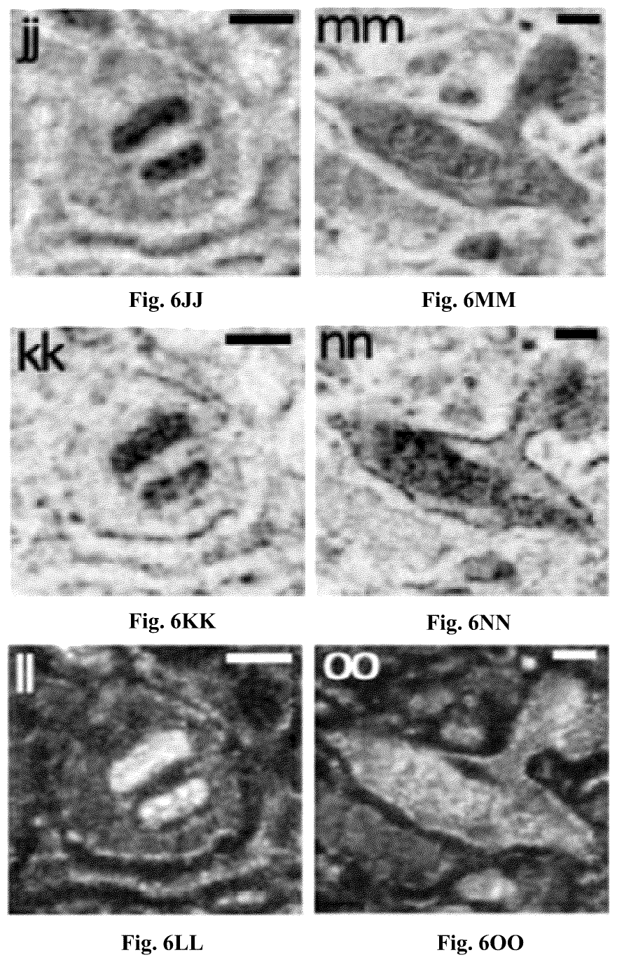

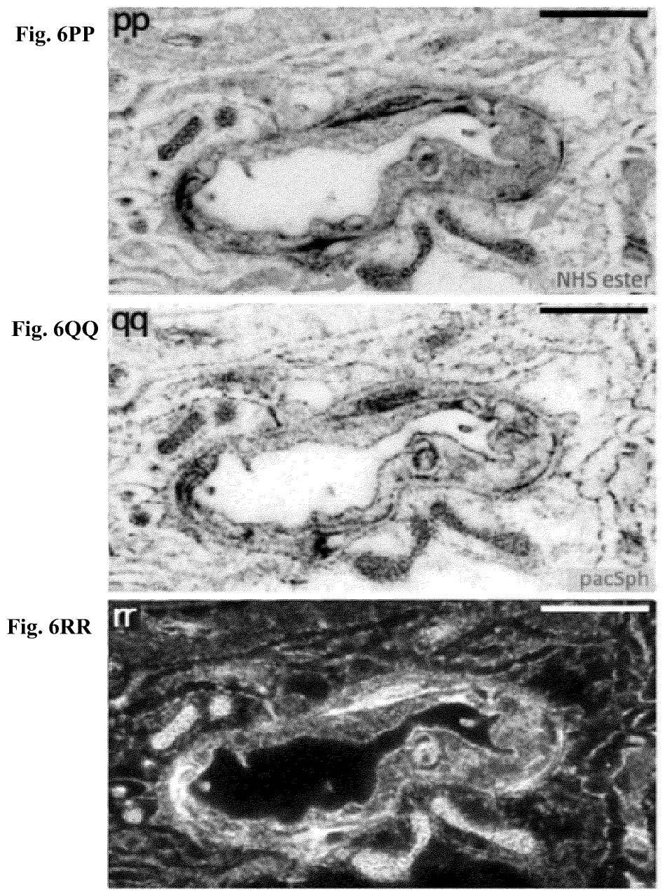

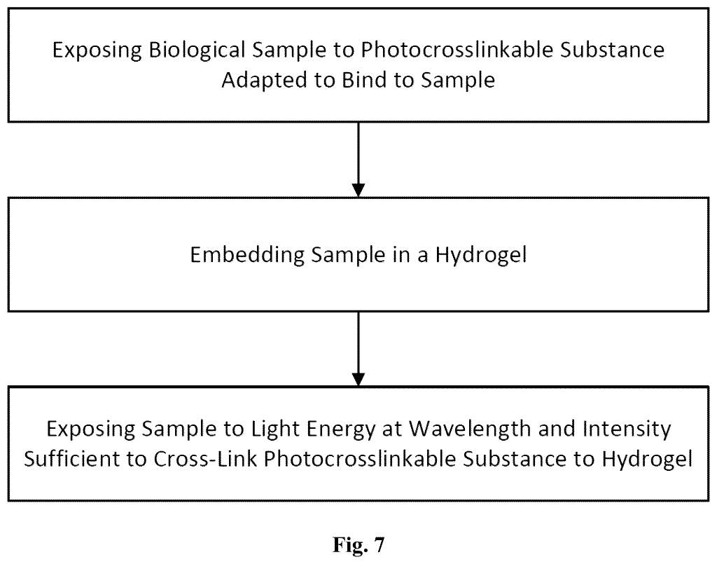

pacSph pan-staining signal in mitochondria was measured by recording the peak intensity of ATTO590 signal from 10-pixel thick line profiles drawn across the width of mitochondria in FIJI. For noUV measurements, 35 peak intensities were recorded from 5 FOVs in one independent experiment. For UV-before measurements, 37 peak intensities were recorded from 5 FOVs in one independent experiment. For UV-after measurements, 39 peak intensities were recorded from 5 FOVs in one independent experiment. Results are summarized in FIG. 6m.

Quantification and statistical analysis

For all quantitative experiments, the number of samples and independent reproductions are listed in the figure legends. An unpaired two-tailed t-test in GraphPad Prism 9 was used to analyze the data.

### Results

pan-ExM reveals synapse ultrastructure in dissociated neurons

Before experimenting with expanding mouse brain tissue sections, Applicant tested pan-ExM in dissociated hippocampal rat and mice neurons using Applicant's published protocol with modifications in sample fixation (see Methods). Applicant sought to test whether pan-ExM in neurons would reveal ultrastructural details previously too crowded or small to be efficiently resolved with conventional microscopy. A visual comparison of NHS ester bulk amine pan-staining of neurons that are non-expanded (FIG. 2, Panel a) or expanded with pan-ExM, (FIG. 2, Panels d-k), confirms the validity of this approach: non-expanded synapses show essentially uniform staining, revealing little information, whereas expanding neurons ˜16-fold allows to spatially resolve synapses by their protein density patterns. Analogous to phosphotungstic acid-(PTA-) staining of neurons in EM (FIG. 2, Panel l), now resolvable hallmark features such as dense projections (DP) of the presynaptic bouton (FIG. 2, Panel m, lime arrow) and the postsynaptic density (FIG. 2, Panel m, salmon arrow) allow for the identification of synapses by their morphological characteristics. Spines that are cupboard shaped (FIG. 2, Panels h,j), thin (FIG. 2, Panels g,k), and stubby (FIG. 2, Panel f) are visible. Strikingly, Applicant also observes triagonal protein-dense patterns formed by presynaptic DPs (FIG. 2, Panel e), a discovery made using freeze-etched replica EM in 1972.

To determine the achieved linear expansion factors, Applicant imaged SYTOX Green-stained neuron nuclei in non-expanded samples and samples expanded using Applicant's standard protocol and compared the average nuclear cross-sectional area in both cases. On average, Applicant obtained an expansion factor of 15.7±0.3 (mean±s.d.; N=4 experiments; n=6-13 nuclei per experiment). Dividing the measured distances between dense projections (DP) and the postsynaptic density (PSD) by the expansion factor determined from nuclei measurements, Applicant obtained a value of 81.9±25.8 nm (mean±s.d.; N=4 experiments; n=44 synaptic profiles; FIG. 2n), which is consistent with the range of pre- and post-synaptic density distance measurements determined previously by super-resolution microscopy and electron tomography. Similarly, dividing the distance between neighboring DPs by the nuclear expansion factor, Applicant obtained a value of 67.2±15.4 nm (mean±s.d.; N=6 experiments; n=78 DP profiles; FIG. 2o), consistent with earlier reports in EM. In all subsequent experiments, Applicant used the DP-PSD distance as a metric for linear expansion factor calculation.

pan-ExM in dissociated neurons is compatible with immunofluorescence labeling as well as other established chemical stainings, enabling correlative studies which combine specific and contextual pan-staining approaches. Focusing on the synapse, FIG. 2, Panels (t-ccc) shows the distributions of synaptic proteins Bassoon, Homer1, PSD-95, and Synaptophysin in the context of synaptic ultrastructure. Applicant observed compartmentalization of active zone protein Bassoon into distinct puncta as dense projections (FIG. 2, Panels t-bb) with synaptic vesicle protein Synaptophysin intercalating in between neighboring dense projections (FIG. 2, Panels xx-ccc), supporting the model that DPs represent distinct sites for synaptic vesicle docking and fusion at the active zone. Applicant also observed nanoclustering of postsynaptic density proteins Homer1 and PSD-95 along an otherwise macular and dense PSD, with Homer1 slightly offsetting the PSD further into the spine (FIG. 2, Panels cc-hh & q) and PSD-95 concentrating directly over the PSD (FIG. 2, Panels ll-qq & r), consistent with previous work. The expansion-corrected distributions of Bassoon, Homer1, and PSD-95 within the axial DP-PSD distances are all in agreement with previously published studies. For instance, the distance between Bassoon and the PSD is 103.7±24.4 nm (mean±s.d.; N=3 independent samples; n=50 synaptic profiles; FIG. 2p); the distance between Homer1 and the DP is 82.86±15.8 nm (mean±s.d.; N=4 independent samples; n=85 synaptic profiles; FIG. 2q); and the distance between PSD-95 and the DP is 68.4±11.0 nm (mean±s.d.; N=2 independent samples; n=25 synaptic profiles; FIG. 2, Panel r). FIG. 2, Panel (s) shows a plot of the positions of Bassoon, Homer1, and PSD-95 along the trans-synaptic axis defined as the center position between DP and PSD.

Double immunostainings are compatible with pan-ExM. FIG. 2, Panels (rr-ww) show Bassoon-Homer1 immunofluorescence images at super-resolution and in their synaptic context. This richness of information is inaccessible with conventional confocal microscopy of unexpanded or only ˜5-fold expanded samples.

Optimizing antibody labeling parameters to achieve efficient and background-free staining is critical for pan-ExM imaging. Because the hydrogels used in pan-ExM are entangled and dense (˜30% w/v monomer concentration), Applicant suspect that many antibodies can become entrapped within this hydrogel matrix when used in high concentrations, often producing a granular background. Applicant found, for example, that lowering the concentrations of both Homer1 primary and dye-conjugated secondary antibodies from ˜2 μg/mL to ˜4 μg/mL strongly reduced the background without compromising signal levels.

Pan-ExM can clearly resolve subcellular structures in dissociated neurons that were previously inaccessible with standard confocal microscopy. FIG. 2, Panels (ddd-fff) shows mtDNA inside a mitochondrion with visible cristae, FIG. 2, Panels (ggg-hhh) shows the hollow, circular structure of a nuclear pore complex, and FIG. 2, Panels (iii-jjj) reveals the cartwheel structure of basal bodies, their distal appendages, and the ciliary tip of a cilium. Furthermore, by combining NHS ester pan-staining with metabolic incorporation of palmitic acid azide, it becomes possible to examine the contact sites of membranous organelles, such as the tubules of the endoplasmic reticulum (ER) and mitochondria.

Pan-ExM-t reveals tissue ultrastructural features

Having established pan-ExM in dissociated neuron cultures, Applicant adapted the technique to 70 μm-thick mouse brain tissue sections. Because of stark differences in thickness, lipid content, and presence of a highly connected extracellular matrix in tissue that is absent in dissociated neurons, brain fixation, sample denaturation, and antibody labeling parameters had to be optimized.

Ultrastructural analysis of brain tissue sections in EM has typically relied on formaldehyde (FA) and glutaraldehyde (GA) fixation to best preserve fine structures. Because pan-ExM is an ultrastructural imaging method, structural preservation at the 10-nm scale is of utmost importance. However, compatibility with immunolabeling is equally important to the core concept of Applicant's technique, and antigens are known to be masked by glutaraldehyde fixation. To examine the effects of fixation and post-fixation on tissue preservation, Applicant expanded 70 μm-thick mouse brain tissue sections that were fixed with 4% FA and not post-fixed (Fix-1), fixed with 4% FA and post-fixed with 0.7% FA+1% acrylamide (AAm) (Fix-2), fixed with 4% FA and post-fixed with 4% FA+20% AAm (Fix-3), fixed with 4% FA+0.1% GA and post-fixed with 4% FA+20% AAm (Fix-4), and fixed with 4% FA and post-treated with 0.1 mg/mL acryloyl-X SE (AcX) (Fix-5). Results suggest that the expansion factor increases with lower post-fixation strength and decreases with the addition of GA in the fixative. They also suggest that using AcX, a common acryloylated and amine-reactive reagent in ExM, results in lower expansion factors as well as multiple artifacts in tissue ultrastructure when coupled with nonenzymatic homogenization.

Moreover, to examine tissue preservation further, Applicant determined the extracellular space and lipid membrane (ECS+) fraction of neuropil across the different fixation schemes presented. The ECS fraction in live organotypic brain slices, determined by STED microscopy, ranges from 5% to 36%. Applicant therefore expects the ECS+fraction to be within that range or higher by ˜10% to account for unlabeled lipid membrane boundaries. It is worth noting that in EM of chemically fixed tissue, the ECS fraction is notoriously underestimated because of tissue shrinkage from excessive fixation (ECS shrinkage is ˜6 fold), giving the false notion that neurites are tightly apposed to one another.

Strong post-fixation with FA and AAm (Fix-3 and Fix-4) is advantageous for good neuropil and ECS preservation. However, these still cause artifactual gaps in neuropil, and in the case of FA+GA-fixed tissue, result in a low expansion factor of ˜11 fold. Therefore, Applicant hypothesized that excessive interprotein crosslinking occurs in the initial fixation stage and predicted that transcardial perfusion with both FA and AAm along with post-fixation in the same fixative would result in a more uniform expansion. Inspired by CLARITY and MAP where FA is combined with acrylamide to simultaneously quench inter-protein crosslinking and functionalize proteins with an AAm group, Applicant used 4% FA+20% AAm in both the transcardial perfusion and overnight post-fixation solutions (Fix-6). Applicant obtained an expansion factor of 24.1±1.4-fold (mean±s.d.; N=10 fields of view from 3 independent experiments; n=254 synaptic profiles, FIG. 3, Panel i) as well as good neuropil preservation, very little artifactual tissue perforations (FIG. 3, Panel a-c), and an acceptable ECS+fraction of ˜38% (FIG. 3, Panel j-l. Applicant therefore decided to use this fixation strategy for all subsequent experiments.

In the process of optimizing protein denaturation conditions, Applicant found that increasing the denaturation temperature above 90° C. or using alkaline buffers resulted in hydrogel disintegration, possibly because of the lability of the DHEBA crosslinker molecule. Applicant therefore adopted the pH 6.8 SDS buffer of pan-ExM and used 75° C. as the temperature for denaturation, similar to 73° C. used in the original protocol. Applicant also found that tissue-hydrogel hybrids denatured for 4 h were macroscopically flat, while gels denatured for fewer than 4 h exhibited crumbled macrostructures, signifying incomplete mechanical homogenization. Applicant therefore assessed denaturation times of 4 h (Denat-4), 6 h (Denat-6), and 8 h (Denat-8) for both expansion factor and relative protein retention (measured by reporting peak DP intensity). Applicant found that Denat-4 results in an expansion factor of 26.9±1.2 (mean±s.d.; N=3 fields of view; n=50 synaptic profiles) and DP peak intensity of 1506.8±243.8 (mean±s.d.; N=3 fields of view; n=50 intensity measurements), Denat-6 yields a similar expansion factor of 26.0±1.3 (mean±s.d.; N=3 fields of view; n=42 synaptic profiles) and slightly lower DP peak intensity of 1353.3±248.3 (mean±s.d.; N=3 fields of view; n=42 intensity measurements), and finally Denat-8 shows an increase in expansion factor to 33.7±1.6 (mean±s.d.; N=3 fields of view; n=100 synaptic profiles) and partial protein loss with DP peak intensity of 1014.3±209.1 (mean±s.d.; N=2 fields of view; n=48 intensity measurements). Since Applicant prioritizes protein retention and argue that expansion factors of ˜24 fold are sufficient to resolve ultrastructural features of interest, Applicant denatured samples for 4 h at 75° C. in all subsequent experiments.

Equipped with a pan-ExM-t protocol that preserves ultrastructure well and allows for ˜24-fold linear expansion, Applicant imaged a wide variety of tissue nanostructures across both hippocampal and cortical regions of the mouse brain. FIG. 3, Panel (a) shows a tiled ˜1×1 mm2 image (corresponding to 42×42 μm2 after correction for the expansion factor) of NHS ester pan-stained cortical tissue at synaptic resolution. Hallmark synaptic features such as pre- and postsynaptic densities can now be imaged and with a standard confocal microscope deep inside a brain tissue section (FIG. 3, Panels d-h). For example, Applicant could observe that putative excitatory synapses , defined by a prominent PSD (FIG. 3, Panels m-cc), often featured mitochondria in the vicinity of axonal boutons (FIG. 3, Panel n, arrow) and sometimes in the postsynaptic partner (FIG. 3, Panel o, arrow). Intriguingly, Applicant can discern densely stained stacked structures in some postsynaptic compartments, suggestive of spine apparatuses (FIG. 3, Panels dd-ii). Moreover, the obtained resolution allowed us to classify synapses based on variations in PSD patterns and intensities into three classes inspired by the previously established Gray classification of synapses: Class 1, where the PSD is dense and macular (FIG. 3, Panel jj); Class 2, where the PSD is dense and perforated (FIG. 3, Panel kk); and Class 3, where the PSD is barely visible, despite prominent DPs in the presynaptic bouton (FIG. 3, Panel 11). Furthermore, pan-ExM-t images clearly reveal the spine neck (FIG. 3, Panel q, arrow) and multisynaptic boutons (FIG. 3, Panel mm-vv).

Next to synapses, centrioles in the perikarya are distinguished by a bright NHS ester pan-staining showing clear distal appendages (FIG. 3, Panel ww) and, in top views, the cartwheel structure (FIG. 3, Panel xx) and nine-fold symmetry (FIG. 3, Panel zz). The nine-fold symmetry is also visible in multiciliated ependymal epithelia lining the lateral ventricles of the mouse brain

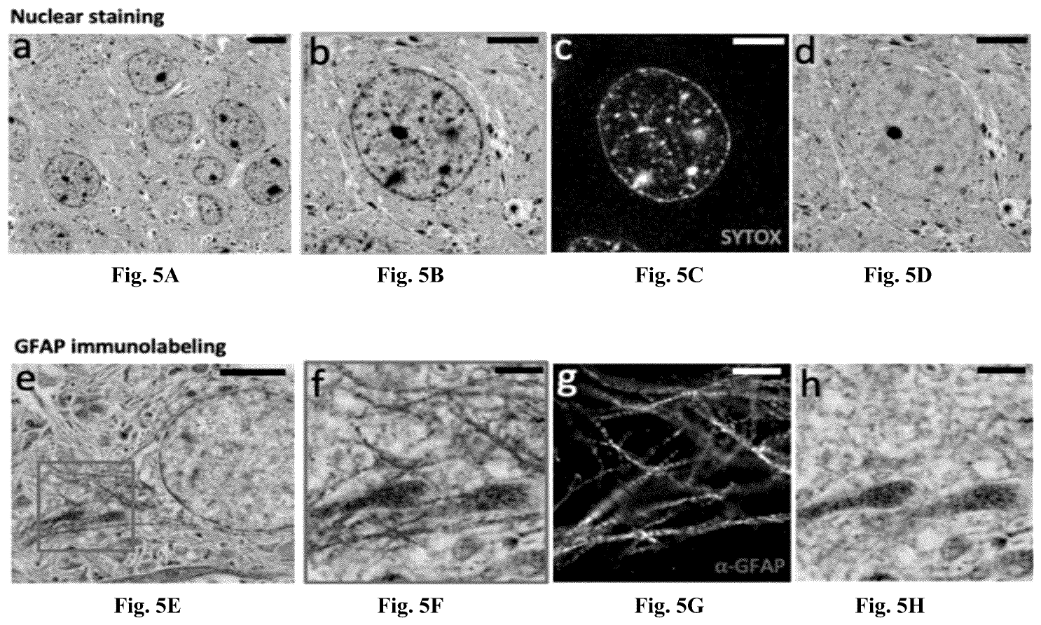

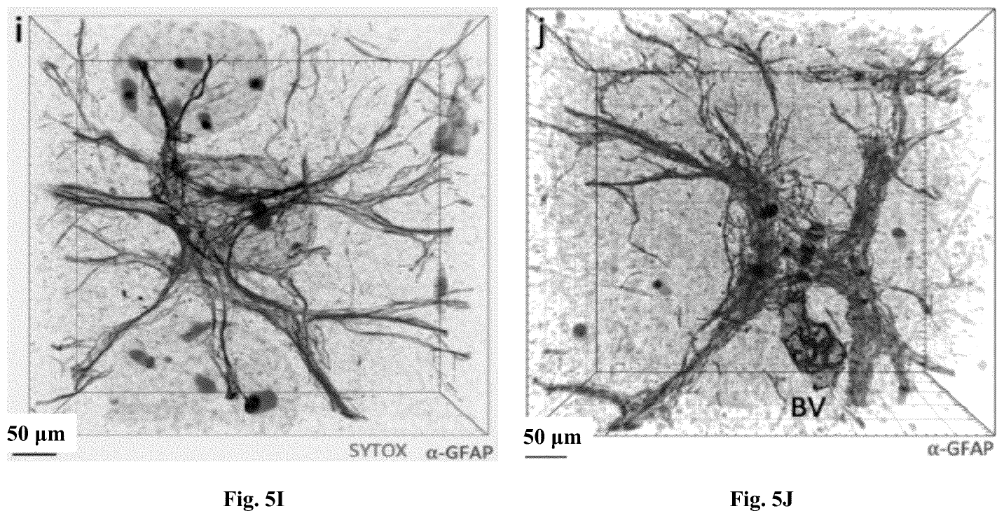

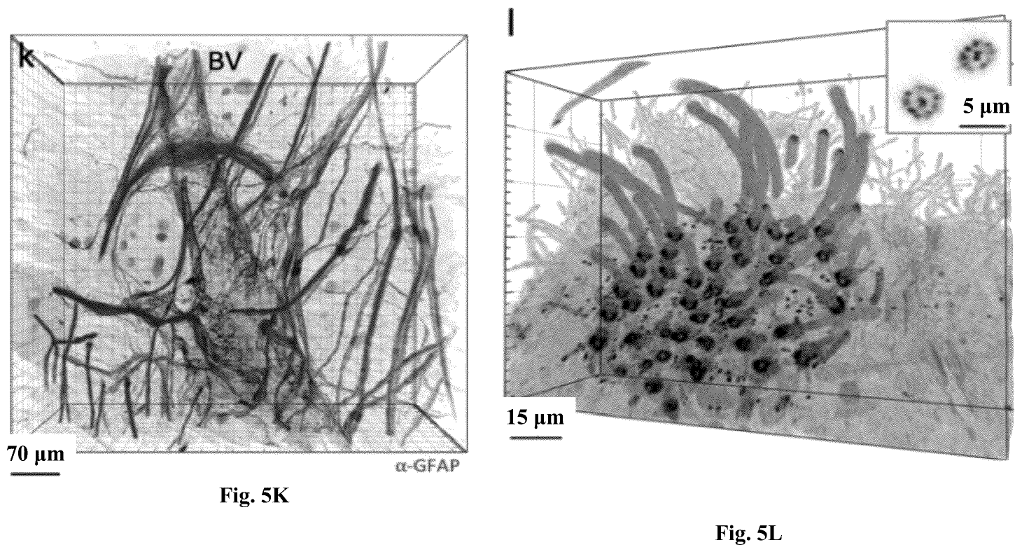

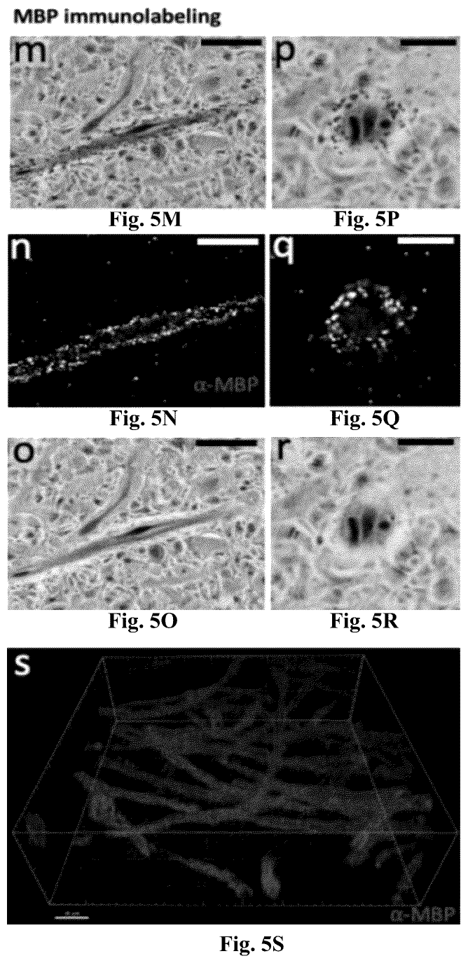

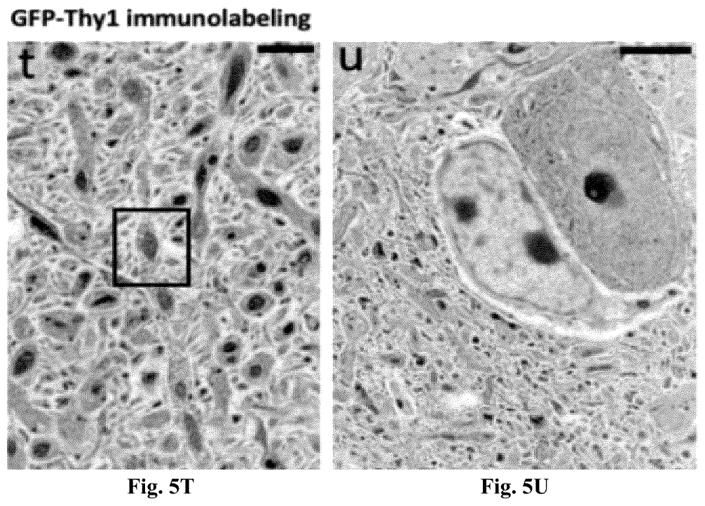

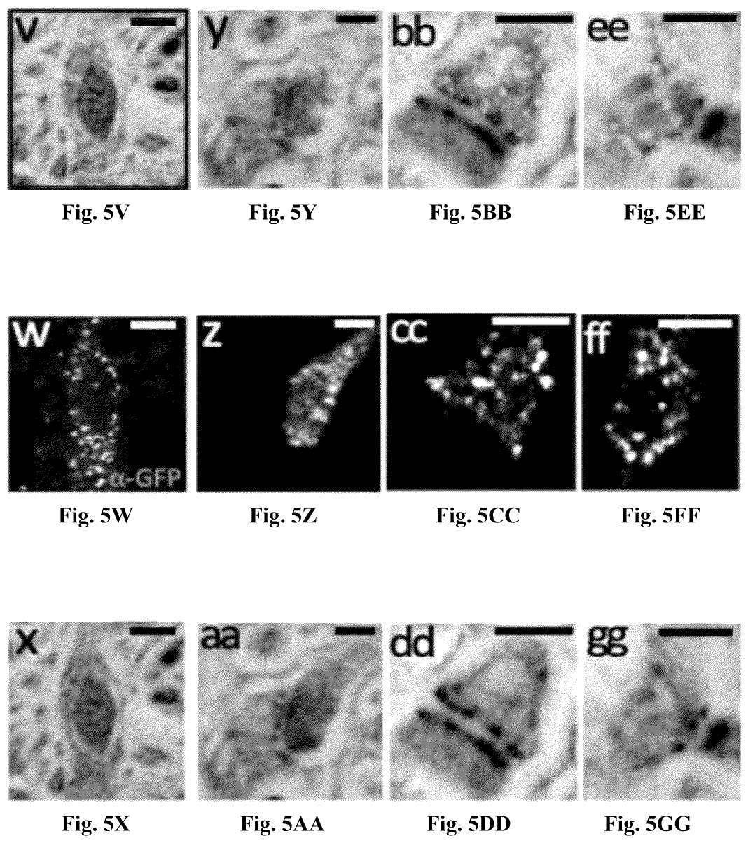

(FIG. 5, Panel 1). Moreover, Applicant also observed that mitochondria morphologies vary strongly across neuropil (FIG. 3, Panels zz-ooo), some featuring clearly resolvable lamellar cristae, and shapes ranging from vesicular to teardrops with tubular extensions (FIG. 3, Panels mmm-ooo, arrows), reported to correlate with disease and synaptic performance. A closer look at brain capillaries (FIG. 3, Panel ooo) reveals clearly discernible endothelial cells, tight junctions, pericytes, and the basement membrane.

pan-ExM-t is compatible with antibody labeling of synapses in thick brain tissue sections

A particular strength of pan-ExM-t is its ability to localize specific proteins to sub-compartments revealed in the contextual pan-stained channel. The protocol therefore allows for efficient antibody staining, while also maintaining good ultrastructural preservation. Because hydrogels with high monomer concentrations are known to impede antibody diffusion, Applicant investigated whether lowering the monomer concentration in the different interpenetrating hydrogels would result in better signal-to-noise antibody stainings. Applicant assessed both antibody-labeling efficiency and ultrastructural preservation in hydrogels synthesized with different sodium acrylate (SA) monomer concentrations. Applicant hypothesized that lowering this concentration would allow for larger effective hydrogel mesh sizes and therefore better antibody penetration. In summary, Applicant used four different hydrogel monomer combinations with either 19% (w/v) or 9% (w/v) SA in the first and second expansion hydrogels, referred to as 19SA/19SA, 19SA/9SA, 9SA/19SA, and 9SA/9SA. Comparing expansion factors, Applicant found that 19SA/19SA gels expand as Applicant previously reported 24.1±1.4-fold (mean±s.d.; N=10 fields of view from 3 independent experiments; n=254 synaptic profiles), 19SA/9SA gels expand 20.3±1.2-fold (mean±s.d.; N=12 fields of view from 2 independent experiments; n=267 synaptic profiles), 9SA/19SA gels expand 20.5±1.8-fold (mean±s.d.; N=6 fields of view from 2 independent experiments; n=88 synaptic profiles), and 9SA/9SA gels expand 18.5±1.7-fold (mean±s.d.; N=6 fields of view from 2 independent experiments; n=86 synaptic profiles). While expansion factors are not radically affected by variations in SA monomer concentrations, Applicant noticed visible ultrastructural differences. Judging tissue preservation by the frequency of gaps in neuropil and detachments of cell bodies from the underlying tissue, Applicant observe equally good tissue integrity in 19SA/19SA, 19SA/9SA, and 9SA/19SA gels, but significantly distorted neuropil and dissociated cell bodies 9SA/9SA gels. Applicant suspects that this distortion effect is due to a decrease in hydrogel mechanical sturdiness when hydrogels of lower monomer concentrations, like the ones in ExR, are used. As for antibody-staining efficiency, Applicant observed equally efficient labeling in 19SA/19SA, 19SA/9SA, and 9SA/19SA hydrogels for most antigens, with reduced background following synaptic protein immunolabeling in 19SA/9SA hydrogels. Therefore, in all subsequent immunoassays, Applicant used 19SA/9SA gels for synaptic protein immunolabeling and 19SA/19SA gels in immunolabeling structural markers.

With a now robust pan-ExM-t protocol capable of localizing specific proteins in their tissue ultrastructural context, Applicant performed several immunostainings against commonly studied synaptic targets. FIG. 4 shows the distributions of synaptic proteins Homer1, Bassoon, PSD-95, and Synaptophysin in the context of tissue and synaptic ultrastructure. The expansion-corrected distributions of Homer1, PSD-95, and Bassoon within the axial DP-PSD distances are all in agreement with published values as well as with values obtained from cultured neuron measurements in this work. For example, the distance between Bassoon and the PSD is 93.7±16.9 nm (mean±s.d.; N=3 fields of view; n=74 synaptic profiles; FIG. 4, Panel ooo); the distance between Homer1 and DP is 93.3±16.1 nm (mean±s.d.; N=5 fields on views; n=120 synaptic profiles; FIG. 4, Panel ppp); and the distance between PSD-95 and the DP is 76.4±11.8 nm (mean±s.d.; N=3 fields of view; n=113 synaptic profiles, FIG. 4, Panel qqq). FIG. 4, Panel (rrr) shows a plot of the axial positions of Homer1, PSD-95, and Bassoon along the trans-synaptic axis defined as the center position between DP and PSD. In agreement with previous work, Homer1 is on average localized further inside the spine and away from the cleft than the PSD center, while PSD-95 is concentrated directly onto the PSD. Interestingly, in Class 3 synapses, defined by the absence of a protein-dense PSD, Applicant found that while Bassoon scaffold protein is present (FIG. 4, Panels q-s) PSD proteins Homer1 (FIG. 4, Panels jj-ll) and PSD-95 (FIG. 4, Panels ddd-fff) are not, reminiscent of inhibitory synapses. pan-ExM-t is compatible with antibody labeling of tissue structures pan-ExM-t is not limited to providing context to synaptic proteins. Applicant found that

SYTOX Green produces a bright nuclear staining that overlays perfectly with NHS ester pan-stained cell nuclei (FIG. 5, Panels a-d). Furthermore, Applicant can resolve individual glial fibrillary acidic protein (GFAP) filaments in astrocytes (FIG. 5, Panels e-k) including near multiciliated ependymal epithelia and engulfing blood vessels (FIG. 5, Panel k. Interestingly, the distinctive pan-stain pattern of astrocyte nuclei and their thick and fibrous cytoplasmic branches allow for their identification without GFAP, analogous to heavy metal-stains in EM. Applicant also observed that myelin basic protein (MBP-) labeled structures are devoid of surrounding pan-staining, suggesting these represent myelinated axons (FIG. 5, Panels m-s). Moreover, when Applicant immunolabeled GFP in Thy1-GFP transgenic mice, Applicant noticed the sparsity of this marker in both neuropil and neuron somas (FIG. 5, Panel t-ii). Originally designed to resolve individual dendrites and axons in densely packed neuropil, GFP-Thy1 imaged with pan-ExM-t reveals these transgenic neurons in their ultrastructural context. pan-ExM-t clearly shows the GFP surrounding, but being excluded from, mitochondria (FIG. 5, Panels v-x). The ability to distinguish GFP-positive dendritic spine heads (FIG. 5, Panel y-aa) and axonal boutons (FIG. 5, Panels bb-gg) from their synaptic partners suggests that pan-ExM-t is especially suited for light-based neural tracing and connectomics.

PacSph enables lipid labeling of tissue in pan-ExM-t

Neural connectomics analysis in EM generally relies on contrast from lipid membranes to delineate cellular boundaries. One way to achieve this in pan-ExM-t would be to preserve the lipid content. Since there are roughly 5 million lipids per 1 μm2 of membrane surface and 150-times more lipids than proteins on a typical cell membrane, Applicant expected that even following detergent extraction with SDS, a fraction of these lipids would remain in the hydrogel. Interestingly, when Applicant labeled SDS-denatured and ˜4-fold expanded brain tissue with BODIPY TR Methyl Ester (BDP), Applicant achieved a distinct pan-staining. Here, BDP highlights the nuclear envelope, organelles like mitochondria, the Golgi complex, the ER, as well as myelinated axons. All these structures resemble those shown in mExM, but were acquired without using any specialized probes. Overlaid with sulfonated NHS ester dye pan-staining, Applicant observed a striking differential hydrophilic-lipophilic pan-staining pattern, with significantly higher BDP staining levels achieved in tissue fixed with 4% FA+0.1% GA than in tissue fixed with 4% FA. Similarly, in pan-ExM-t expanded tissue, Applicant achieved distinctive and bright BDP labeling in tissue fixed with 4% FA+0.1% GA, but almost no staining in tissue fixed with 4% FA. Applicant also see major structural artifacts when supplementing the fixatives with 0.01% osmium tetroxide, known to better preserve lipids. These results confirm that GA plays a major role in stabilizing and crosslinking the lipid content, and suggest that monoaldehydes like FA alone do not provide adequate lipid preservation when preceded by detergent extraction.

Next, Applicant asked whether replacing the anionic detergent SDS in Applicant's denaturation buffer with the chaotropic reagent guanidine hydrochloride (G-HCl) would better preserve the lipid content and enable superior lipid staining in 4% FA+0.1% GA-fixed, pan-ExM-t expanded tissue. Unlike SDS, hyperhydration with urea or guanidine HCl is known to disrupt hydrogen bonding in proteins without solubilizing lipids. EM images of brain tissue samples fixed with only 4% FA and chemically cleared with the functionally similar chaotropic molecule urea show intact membrane ultrastructure and up to 70% lipid retention. Applicant chose to work with G-HCl and not urea, because the latter is known to decompose and carbamylate proteins at the elevated temperatures required for efficient denaturation. BDP signal is in fact higher in G-HCl-denatured neuron and tissue samples, giving credence to this approach. However, the data also show that BDP does not exclusively stain cell membranes and subjecting samples to no denaturation at all results in lower BPD signal. This observation is more evident in ˜20-fold expanded samples where easily distinguishable neurite boundaries are not specifically stained with BDP. All these results suggest that lipophilic dyes like BDP do not exclusively label lipid membranes in denatured samples, but also label the hydrophobic core of proteins exposed following denaturation. In fact, in thermal denaturation assays, environmentally sensitive fluorescent dyes that bind to the hydrophobic core of proteins are commonly used to determine the temperature at which proteins denature (e.g., in differential scanning fluorimetry (DSF)). Protein denaturation, however, is a necessary step in enzyme-free ExM methods, since electrostatic interactions between proteins must be nulled for isotropic sample expansion.

Since strategies to preserve the lipid content for post-homogenization lipophilic labeling do not result in exclusive cell membrane delineation, and require antigen-masking GA fixation, Applicant decided to capitalize on pre-denaturation lipid labeling strategies. Here, proteins are not unfolded, and the lipid content may require less harsh fixation since one does not need to preserve it after the homogenization process. To label lipid membranes in ExM, all approaches have so far used trifunctional lipid probes in either live or fixed samples. In these probes, one functional group is a membrane-intercalating lipid, the second is hydrogel-anchorable, and the third is a reporter molecule. In the live-labeling approaches, cells have been labeled with acryolated and fluorescent 1,2-distearoyl-sn-glycero-3-phosphoethanolamine (DSPE), clickable palmitic acid, aminated α-NH2-ω-N3-C6-ceramide, or clickable choline. The only strategy available to stain lipids in tissue with ExM after it is fixed is to use aminated (poly-lysine) membrane-intercalating probes such as pG5kb and mCling. These survive detergent disruption by covalent attachment to the hydrogel matrix and contain a biotin group available for conjugation with dye-labeled streptavidin after expansion. When Applicant tested mCling in 5-fold expanded brain tissue sections, Applicant found that although it labeled tissue hydrophobic components, this labeling was limited to the tissue surface, likely because of diffusion limitations. When Applicant tested mCling in pan-ExM-t samples, Applicant observed little to no labeling. mCling as well as an ExM-optimized version of it, pG5kb, have 5 to 7 lysine groups making them relatively highly charged and bulky. mCling and pG5kb also require that the tissue is fixed with GA, which Applicant previously found decreases synaptic protein antigenicity as well as limits the expansion factor to ˜12 fold.

Applicant therefore developed a new pan-ExM-t lipid labeling strategy using a commercially available photocrosslinkable and clickable sphingosine pacSph as a lipid intercalating reagent in tissue fixed only with 4% FA+20% AAm (to preserve antigenicity) (FIG. 6, Panel n). In the protocol, Applicant (1) labeled 70 μm-thick tissue sections with pacSph prior to hydrogel embedding; (2) irradiated the sample with UV light (365 nm) to photo-crosslink the probes onto neighboring proteins (from both directions to compensate for depth-dependent UV light absorption in the 70 μM thick tissue); and (3) probed the ligands with fluorescent dyes after homogenization and expansion. It is worth noting that pacSph is smaller (335 Da) than mCling (1445 Da) or pG5kb (1158 Da), polar, and partially positively charged (amine pKa ˜6.6), allowing for high probe packing and sampling. Sphingosines are also membrane bilayer-rigidifying: they stabilize gel domains in membranes, raise their melting temperature, and induce membrane permeabilization without micellar extraction of proteins or lipids. Applicant therefore expects sphingosine to diffuse efficiently in thick tissue. Furthermore, pacSph has a photoactivatable diazirine group that forms reactive carbene intermediates capable of insertion in C—H or N—H, and O—H bonds of tissue components upon UV irradiation. Since diazirine is on the lipid chain (FIG. 6, Panel n, pink diazirine), Applicant expects that two pacSph lipids located on opposing sides of a membrane bilayer will photo-crosslink the embedded hydrogel polymer chains at the center of the hydrophobic phase of the bilayer, minimizing the linker error. This error is inevitable if onewere to photo-crosslink the probes at the polar head group. Finally, retained lipids are conjugated with a hydrophobic azido dye at the lipid tail extremity (FIG. 6n, green alkyne). Applicant believe these factors make pacSph ideally suited for membrane delineation in pan-ExM-t.

FIG. 6 confirms the strength of the approach. In the perikarya of brain tissue fixed with 4% FA+20% AAm and pan-stained with pacSph, one can clearly discern both sides of ER tubules (FIG. 6, Panel d-f), ER and mitochondria contact sites (FIG. 6, Panels g-i), and both sides of the nuclear envelope (FIG. 6, Panels j-1). In neuropil, lipid membrane boundaries are now discernable (FIG. 6, Panels o-oo), revealing the boundaries of neurites and synaptic compartments. In brain capillaries, the different membranes are also differentially highlighted relative to NHS ester pan-staining (FIG. 6, Panels pp-rr). Moreover, when Applicant experimented with irradiating pacSph pan-stained tissue with UV light before or after the first hydrogel embedding, Applicant found that UV irradiation after hydrogel embedding results in significantly higher probe retention, suggesting that diazirine is being photo-crosslinked to the dense hydrogel mesh in addition to surrounding proteins (FIG. 6, Panel m). A recent study showed that diazirine has an affinity for reacting with proteins containing large negative electrostatic surfaces in cells (e.g., carboxyl groups). Applicant suspects that diazirine in the probe is preferentially photo-crosslinked to carboxyl groups on the poly(acrylamide/acrylate) mesh, offering better retention of the lipid content. While benefiting from future investigation, the data shows that photo-crosslinking lipid-intercalating ceramides to the expansion hydrogel represents an important strategy in labeling lipids in ˜24-fold expanded and formaldehyde fixed brain tissue sections.

### Discussion

The data presented in this paper demonstrates that pan-ExM-t can clearly resolve molecular targets in the context of brain tissue ultrastructure. By combining ˜24-fold linear sample expansion with novel pan-stainings, Applicant was able to (1) resolve and identify synapses and their substructures by their morphological characteristics, (2) localize protein markers to their subcellular compartments, and (3) delineate cellular membranes.

Pan-ExM-t achieves this level of detail by taking advantage of an opportunity inaccessible to optical super-resolution techniques: the relative size of labels becomes irrelevant since labels appear shrunken by the linear expansion factor and the relative radius of a fluorescent dye (˜1 nm) approaches ˜1 nm/20=˜50 μm, a size comparable to that of an osmium atom used in heavy-metal EM stainings (˜200 μm). The distance between two proteins that originally were in close proximity (˜2 nm) grows to ˜40 nm, well beyond the Förster radius (˜5 nm) of typical fluorescent dyes. Bulk fluorescence staining of decrowded neuropil is therefore no longer limited by size, sampling and quenching restrictions of fluorescent dyes, and ultrastructural details, previously accessible only by EM, can now be examined on standard light microscopes.

This disclosure shows that brain tissue structures such as neurons, glia, axons, dendrites, blood capillaries, pericytes, the endothelium, basal bodies, as well as subcellular features like DPs, PSDs, mitochondria cristae, ER tubules, myelin sheaths, multisynaptic boutons, spine necks, spine apparatuses, and the cartwheel structure of centrioles can all be identified in the pan-stain channel alone, analogous to classical EM techniques. Applicant also demonstrated, for the first time, that chemical synapses can be distinguished in a light microscopic image by their characteristic protein density patterns. These patterns could be characterized analogously to the Gray classification established for PTA and osmium stained synapses in EM. Taking advantage of pan-ExM-t's strength of directly correlating synapse ultrastructure with immunolabeling against specific proteins, Applicant observed that Class 1 and Class 2, but not Class 3, synapses are positive for Homer1 and PSD-95, supporting the functional Gray synapse concept that synapses with dense PSDs are likely excitatory. This example demonstrates the approach's capability to map the molecular makeup of different synapse types within the context of their distinct ultrastructural properties, which could lead to a systematic exploration of synapse diversity and a determination of synapse sub-types that are vulnerable to diseases.

To achieve the highest ultrastructural preservation of tissue while maximizing sample expansion, Applicant showed that it is important to optimize fixation and gelation parameters. NHS ester pan-staining in this context serves as a readily accessible readout to assess the preservation and retention of proteins. Applicant demonstrated that reducing interprotein crosslinking enables 26-fold expansion without resorting to protease treatments or harsh denaturation conditions. Applicant also demonstrated that hydrogels of high monomer concentrations (˜30% w/v) and sufficient crosslinker density (˜0.1% w/v) must be used to ensure that proteins are adequately sampled by the polymer matrix. In this context, it is not surprising that pan-ExM-t gels are stiffer than the gels prepared using the standard ExM protocol. Moreover, Applicant found that ECS preservation is sensitive to the strength of fixatives used, with higher fixation resulting in lower ECS fractions. In all cases, the ECS is significantly more prominent in pan-ExM-t than in EM, emphasizing that the familiar appearance of tightly apposed neurites, typically seen in EM, is artifactual, and consistent with a previous live-STED microscopy characterization of brain ECS.

In this work, Applicant also explored the feasibility of imaging lipid membranes in 24-fold expanded brain tissue sections. Applicant reasoned that attempts to preserve the endogenous lipid content and probe it after expansion are counterproductive. After all, since the free energy of lipid bilayers must be minimized, Applicant hypothesizes that even if one were to avoid detergent extraction, lipid membranes will eventually collapse into micelles following sample expansion. The data shows that although lipophilic dyes in protein-denatured samples produce a distinctive pan-stain, these likely also stain the hydrophobic core of proteins. Applicant's strategy was therefore to imprint lipid membranes onto the hydrogel before mechanical homogenization of the sample. Applicant demonstrated here that pacSph, a photocrosslinkable and clickable sphingosine, enables neuronal membrane imaging in tissue fixed only with formaldehyde. Applicant was able to delineate cellular organelles, synaptic anatomy, as well as membrane boundaries. The new contrast complements Applicant's established protein pan-stain, and can potentially facilitate segmentation and neuronal tracing algorithms for connectomics analysis.

Tracing lipid boundaries in neuropil using EM has enabled reconstruction of the neural wiring diagrams of a 1,500-μm³ large volume in the mouse brain, the Drosophila larval mushroom body, and the Caenorhabditis elegans connectome. To date, however, no light microscopy method has been able to reconstruct a brain connectome, as these lack the ability of EM to reveal neurite ultrastructure without selective staining. Interestingly, one theoretical study supports the feasibility of reconstructing entire connectomes using lipid membrane labeling and in-situ molecular barcoding in 20-fold expanded tissue. Based on the data, however, capitalizing on protein (instead of membrane) pan-staining in >20-fold expanded tissue might be a more feasible step towards this goal. The data shows that cell boundaries can be detected easily by the absence of protein pan-staining. In contrast to lipids, which need to be negatively imprinted onto the expansion hydrogel first, proteins can be retained well through the expansion process. While Applicant did not reconstruct neural connectivity in this work, individual neighboring neurites and pre- and post-synaptic compartments can be resolved relatively easily, laying the groundwork to computational tracing of neurons. Applicant envisions that combining pan-ExM-t with microscopes optimized for high-resolution and large field of view imaging with molecular optical barcoding techniques that are nucleic acid- or protein-based will revolutionize this active area of research.

Similar to the early days of EM, Applicant believes that many future optimizations will enhance this work. Sample preservation could be further improved, lipid membrane delineation would benefit from systematic optimizations, and more experiments could be designed to characterize the multitude of ultrastructural features revealed by the presented pan-stainings. However, the core concept holds true regardless: that with adequate sample preservation and expansion, pan-staining brings light microscopy to the realm of ultrastructural context imaging.

No longer in the dark, Applicant now see the brain in its complex makeup and the totality of its diverse neural processes: from the microstructure of cells and neuropil down to the nanoarchitecture of synaptic densities. No longer in the void, fluorescing antigens are now localized to their nanoscopic compartments, providing a functional dimension to the underlying structures they embody.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method for preparing a biological sample, the method comprising:
exposing the biological sample to a photocrosslinkable substance adapted to bind to the biological sample, thereby forming a plurality of sample-photocrosslinkable-substance conjugates;
embedding the sample in a hydrogel; and
exposing the sample to light energy at a wavelength and intensity sufficient to cross-link the photocrosslinkable substance to the hydrogel.

2. The method of claim 1, wherein the photocrosslinkable substance is of X-Y-Z format wherein X can bind a biological substance, Y is a photocrosslinkable group, and Z is a reporter group.

3. The method of claim 1, wherein the photocrosslinkable substance comprises diazirine.

4. The method of claim 1, wherein the photocrosslinkable substance comprises one or more selected from the group consisting of: a reporter group, a Clickable reporter group, and an alkyne reporter group.

5. The method of claim 1, wherein the light energy is UV light.

6. The method of claim 1, wherein the light energy has a wavelength between 200 and 600 nm.

7. The method of claim 1, wherein the hydrogel is swellable.

8. The method of claim 1 wherein the hydrogel is swellable and the hydrogel is modified by inserting additional molecules into the hydrogel to increase its size.

9. The method of claim 1, wherein the sample is photocrosslinked after embedding in a hydrogel.

10. The method of claim 1, wherein the sample is photocrosslinked before polymerization of the hydrogel.

11. The method of claim 1, wherein the sample is photocrosslinked while being embedded in a hydrogel.

12. The method of claim 1, wherein the sample is photocrosslinked to one or more selected from the group consisting of: a first and a second hydrogel network in a double-network hydrogel.

13. The method of claim 1, wherein the sample is photocrosslinked to one or more selected from the group consisting of: a first and an nth hydrogel network in a multiple-network hydrogel, wherein n is a positive integer greater than 2.

14. The method of claim 1, wherein the photocrosslinkable substance binds to proteins.

15. The method of claim 1, wherein the photocrosslinkable substance comprises an amine reactive group.

16. The method of claim 1, wherein the photocrosslinkable substance comprises an NHS ester group.

17. The method of claim 1, wherein the photocrosslinkable substance binds to nucleic acids.

18. The method of claim 1, wherein the photocrosslinkable substance binds to lipids.

19. The method of claim 1, wherein the photocrosslinkable substance comprises a lipid.

20. The method of claim 1, wherein the photocrosslinkable substance binds to carbohydrates.
